SEQUENCE LISTING (87 sequences)

SEQ ID NO: 1 - DNA, 15 bp, Artificial Sequence
  Synthetic (DNAzyme)
ggctagctacaacga

SEQ ID NO: 2 - DNA, 6030 bp, Homo sapiens
gttggcccccgttacttttcctctgggaaatatggcgcacgctgggagaacagggtacgataaccgggagatagtgatgaagtacatccattataagctgtcgcagaggggctacgagtgggatgcgggagatgtgggcgccgcgcccccgggggccgcccccgcgccgggcatcttctcctcgcagcccgggcacacgccccatacagccgcatcccgggacccggtcgccaggacctcgccgctgcagaccccggctgcccccggcgccgccgcggggcctgcgctcagcccggtgccacctgtggtccacctgaccctccgccaggccggcgacgacttctcccgccgctaccgccgcgacttcgccgagatgtccaggcagctgcacctgacgcccttcaccgcgcggggacgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggatgcctttgtggaactgtacggccccagcatgcggcctctgtttgatttctcctggctgtctctgaagactctgctcagtttggccctggtgggagcttgcatcaccctgggtgcctatctgggccacaagtgaagtcaacatgcctgccccaaacaaatatgcaaaaggttcactaaagcagtagaaataatatgcattgtcagtgatgttccatgaaacaaagctgcaggctgtttaagaaaaaataacacacatataaacatcacacacacagacagacacacacacacacaacaattaacagtcttcaggcaaaacgtcgaatcagctatttactgccaaagggaaatatcatttattttttacattattaagaaaaaaagatttatttatttaagacagtcccatcaaaactcctgtctttggaaatccgaccactaattgccaagcaccgcttcgtgtggctccacctggatgttctgtgcctgtaaacatagattcgctttccatgttgttggccggatcaccatctgaagagcagacggatggaaaaaggacctgatcattggggaagctggctttctggctgctggaggctggggagaaggtgttcattcacttgcatttctttgccctgggggctgtgatattaacagagggagggttcctgtggggggaagtccatgcctccctggcctgaagaagagactctttgcatatgactcacatgatgcatacctggtgggaggaaaagagttgggaacttcagatggacctagtacccactgagatttccacgccgaaggacagcgatgggaaaaatgcccttaaatcataggaaagtatttttttaagctaccaattgtgccgagaaaagcattttagcaatttatacaatatcatccagtaccttaagccctgattgtgtatattcatatattttggatacgcaccccccaactcccaatactggctctgtctgagtaagaaacagaatcctctggaacttgaggaagtgaacatttcggtgacttccgcatcaggaaggctagagttacccagagcatcaggccgccacaagtgcctgcttttaggagaccgaagtccgcagaacctgcctgtgtcccagcttggaggcctggtcctggaactgagccggggccctcactggcctcctccagggatgatcaacagggcagtgtggtctccgaatgtctggaagctgatggagctcagaattccactgtcaagaaagagcagtagaggggtgtggctgggcctgtcaccctggggccctccaggtaggcccgttttcacgtggagcatgggagccacgacccttcttaagacatgtatcactgtagagggaaggaacagaggccctgggcccttcctatcagaaggacatggtgaaggctgggaacgtgaggagaggcaatggccacggcccattttggctgtagcacatggcacgttggctgtgtggccttggcccacctgtgagtttaaagcaaggctttaaatgactttggagagggtcacaaatcctaaaagaagcattgaagtgaggtgtcatggattaattgacccctgtctatggaattacatgtaaaacattatcttgtcactgtagtttggttttatttgaaaacctgacaaaaaaaaagttccaggtgtggaatatgggggttatctgtacatcctggggcattaaaaaaaaaatcaatggtggggaactataaagaagtaacaaaagaagtgacatcttcagcaaataaactaggaaatttttttttcttccagtttagaatcagccttgaaacattgatggaataactctgtggcattattgcattatataccatttatctgtattaactttggaatgtactctgttcaatgtttaatgctgtggttgatatttcgaaagctgctttaaaaaaatacatgcatctcagcgtttttttgtttttaattgtatttagttatggcctatacactatttgtgagcaaaggtgatcgttttctgtttgagatttttatctcttgattcttcaaaagcattctgagaaggtgagataagccctgagtctcagctacctaagaaaaacctggatgtcactggccactgaggagctttgtttcaaccaagtcatgtgcatttccacgtcaacagaattgtttattgtgacagttatatctgttgtccctttgaccttgtttcttgaaggtttcctcgtccctgggcaattccgcatttaattcatggtattcaggattacatgcatgtttggttaaacccatgagattcattcagttaaaaatccagatggcaaatgaccagcagattcaaatctatggtggtttgacctttagagagttgctttacgtggcctgtttcaacacagacccacccagagccctcctgccctccttccgcgggggctttctcatggctgtccttcagggtcttcctgaaatgcagtggtgcttacgctccaccaagaaagcaggaaacctgtggtatgaagccagacctccccggcgggcctcagggaacagaatgatcagacctttgaatgattctaatttttaagcaaaatattattttatgaaaggtttacattgtcaaagtgatgaatatggaatatccaatcctgtgctgctatcctgccaaaatcattttaatggagtcagtttgcagtatgctccacgtggtaagatcctccaagctgctttagaagtaacaatgaagaacgtggacgcttttaatataaagcctgttttgtcttctgttgttgttcaaacgggattcacagagtatttgaaaaatgtatatatattaagaggtcacgggggctaattgctggctggctgccttttgctgtggggttttgttacctggttttaataacagtaaatgtgcccagcctcttggccccagaactgtacagtattgtggctgcacttgctctaagagtagttgatgttgcattttccttattgttaaaaacatgttagaagcaatgaatgtatataaaagcctcaactagtcatttttttctcctcttcttttttttcattatatctaattattttgcagttgggcaacagagaaccatccctattttgtattgaagagggattcacatctgcatcttaactgctctttatgaatgaaaaaacagtcctctgtatgtactcctctttacactggccagggtcagagttaaatagagtatatgcactttccaaattggggacaagggctctaaaaaaagccccaaaaggagaagaacatctgagaacctcctcggccctcccagtccctcgctgcacaaatactccgcaagagaggccagaatgacagctgacagggtctatggccatcgggtcgtctccgaagatttggcaggggcagaaaactctggcaggcttaagatttggaataaagtcacagaatcaaggaagcacctcaatttagttcaaacaagacgccaacattctctccacagctcacttacctctctgtgttcagatgtggccttccatttatatgtgatctttgttttattagtaaatgcttatcatctaaagatgtagctctggcccagtgggaaaaattaggaagtgattataaatcgagaggagttataataatcaagattaaatgtaaataatcagggcaatcccaacacatgtctagctttcacctccaggatctattgagtgaacagaattgcaaatagtctctatttgtaattgaacttatcctaaaacaaatagtttataaatgtgaacttaaactctaattaattccaactgtacttttaaggcagtggctgtttttagactttcttatcacttatagttagtaatgtacacctactctatcagagaaaaacaggaaaggctcgaaatacaagccattctaaggaaattagggagtcagttgaaattctattctgatcttattctgtggtgtcttttgcagcccagacaaatgtggttacacactttttaagaaatacaattctacattgtcaagcttatgaaggttccaatcagatctttattgttattcaatttggatctttcagggattttttttttaaattattatgggacaaaggacatttgttggaggggtgggagggaggaacaatttttaaatataaaacattcccaagtttggatcagggagttggaagttttcagaataaccagaactaagggtatgaaggacctgtattggggtcgatgtgatgcctctgcgaagaaccttgtgtgacaaatgagaaacattttgaagtttgtggtacgacctttagattccagagacatcagcatggctcaaagtgcagctccgtttggcagtgcaatggtataaatttcaagctggatatgtctaatgggtatttaaacaataaatgtgcagttttaactaacaggatatttaatgacaaccttctggttggtagggacatctgtttctaaatgtttattatgtacaatacagaaaaaaattttataaaattaagcaatgtgaaactgaattggagagtgataatacaagtcctttagtcttacccagtgaatcattctgttccatgtctttggacaaccatgaccttggacaatcatgaaatatgcatctcactggatgcaaagaaaatcagatggagcatgaatggtactgtaccggttcatctggactgccccagaaaaataacttcaagcaaacatcctatcaacaacaaggttgttctgcataccaagctgagcacagaagatgggaacactggtggaggatggaaaggctcgctcaatcaagaaaattctgagactattaataaataagactgtagtgtagatactgagtaaatccatgcacctaaaccttttggaaaatctgccgtgggccctccagatagctcatttcattaagtttttccctccaaggtagaatttgcaagagtgacagtggattgcatttcttttggggaagctttcttttggtggttttgtttattataccttcttaagttttcaaccaaggtttgcttttgttttgagttactggggttatttttgttttaaataaaaataagtgtacaataagtgtttttgtattgaaagcttttgttatcaagattttcatacttttaccttccatggctctttttaagattgatacttttaagaggtggctgatattctgcaacactgtacacataaaaaatacggtaaggatactttacatggttaaggtaaagtaagtctccagttggccaccattagctataatggcactttgtttgtgttgttggaaaaagtcacattgccattaaactttccttgtctgtctagttaatattgtgaagaaaaataaagtacagtgtgagatactg

SEQ ID NO: 3 - DNA, 926 bp, Homo sapiens
gaatctctttctctcccttcagaatcttatcttggctttggatcttagaagagaatcactaaccagagacgagactcagtgagtgagcaggtgttttggacaatggactggttgagcccatccctattataaaaatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactgcgcacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctgctgggctcactcttcagtcggaaatgaccagacactgaccatccactctaccctcccacccccttctctgctccaccacatcctccgtccagccgccattgccaccaggagaacccg

SEQ ID NO: 4 - DNA, 582 bp, Homo sapiens
atggcgaccccagcctcggccccagacacacgggctctggtggcagactttgtaggttataagctgaggcagaagggttatgtctgtggagctggccccggggagggcccagcagctgacccgctgcaccaagccatgcgggcagctggagatgagttcgagacccgcttccggcgcaccttctctgatctggcggctcagctgcatgtgaccccaggctcagcccaacaacgcttcacccaggtctccgatgaactttttcaagggggccccaactggggccgccttgtagccttctttgtctttggggctgcactgtgtgctgagagtgtcaacaaggagatggaaccactggtgggacaagtgcaggagtggatggtggcctacctggagacgcagctggctgactggatccacagcagtgggggctgggcggagttcacagctctatacggggacggggccctggaggaggcgcggcgtctgcgggaggggaactgggcatcagtgaggacagtgctgacgggggccgtggcactgggggccctggtaactgtaggggccttttttgctagcaagtga

SEQ ID NO: 5 - DNA, 780 bp, Homo sapiens
gagtgagcattctcagcacattgcctcaacagcttcaaggtgagccagctcaagactttgctctccaccaggcagaagatgacagactgtgaatttggatatatttacaggctggctcaggactatctgcagtgcgtcctacagataccacaacctggatcaggtccaagcaaaacgtccagagtgctacaaaatgttgcgttctcagtccaaaaagaagtggaaaagaatctgaagtcatgcttggacaatgttaatgttgtgtccgtagacactgccagaacactattcaaccaagtgatggaaaaggagtttgaagacggcatcattaactggggaagaattgtaaccatatttgcatttgaaggtattctcatcaagaaacttctacgacagcaaattgccccggatgtggatacctataaggagatttcatattttgttgcggagttcataatgaataacacaggagaatggataaggcaaaacggaggctgggaaaatggctttgtaaagaagtttgaacctaaatctggctggatgacttttctagaagttacaggaaagatctgtgaaatgctatctctcctgaagcaatactgttgaccagaaaggacactccatattgtgaaaccggcctaatttttctgactgatatggaaacgattgccaacacatacttctacttttaaataaacaactttgatgatgtaacttgaccttccagagttatggaaattttgtccccatgtaatgaataaattgtatgtatttttctct

SEQ ID NO: 6 - DNA, 2430 bp, Homo sapiens
gtcggggtcttccccagttttctcagccaggcggcggcggcgactggcaatgtttggcctcaaaagaaacgcggtaatcggactcaacctctactgtgggggggccggcttgggggccggcagcggcggcgccacccgcccgggagggcgacttttggctacggagaaggaggcctcggcccggcgagagatagggggaggggaggccggcgcggtgattggcggaagcgccggcgcaagccccccgtccaccctcacgccagactcccggagggtcgcgcggccgccgcccattggcgccgaggtccccgacgtcaccgcgacccccgcgaggctgcttttcttcgcgcccacccgccgcgcggcgccgcttgaggagatggaagccccggccgctgacgccatcatgtcgcccgaagaggagctggacgggtacgagccggagcctctcgggaagcggccggctgtcctgccgctgctggagttggtcggggaatctggtaataacaccagtacggacgggtcactaccctcgacgccgccgccagcagaggaggaggaggacgagttgtaccggcagtcgctggagattatctctcggtaccttcgggagcaggccaccggcgccaaggacacaaagccaatgggcaggtctggggccaccagcaggaaggcgctggagaccttacgacgggttggggatggcgtgcagcgcaaccacgagacggccttccaaggcatgcttcggaaactggacatcaaaaacgaagacgatgtgaaatcgttgtctcgagtgatgatccatgttttcagcgacggcgtaacaaactggggcaggattgtgactctcatttcttttggtgcctttgtggctaaacacttgaagaccataaaccaagaaagctgcatcgaaccattagcagaaagtatcacagacgttctcgtaaggacaaaacgggactggctagttaaacaaagaggctgggatgggtttgtggagttcttccatgtagaggacctagaaggtggcatcaggaatgtgctgctggcttttgcaggtgttgctggagtaggagctggtttggcatatctaataagatagccttactgtaagtgcaatagttgacttttaaccaaccaccaccaccaccaaaaccagtttatgcagttggactccaagctgtaacttcctagagttgcaccctagcaacctagccagaaaagcaagtggcaagaggattatggctaacaagaataaatacatgggaagagtgctccccattgattgaagagtcactgtctgaaagaagcaaagttcagtttcagcaacaaacaaactttgtttgggaagctatggaggaggacttttagatttagtgaagatggtagggtggaaagacttaatttccttgttgagaacaggaaagtggccagtagccaggcaagtcatagaattgattacccgccgaattcattaatttactgtagtagtgttaagagaagcactaagaatgccagtgacctgtgtaaaagttacaagtaatagaactatgactgtaagcctcagtactgtacaagggaagcttttcctctctctaattagctttcccagtatacttcttagaaagtccaagtgttcaggacttttatacctgttatactttggcttggttccatgattcttactttattagcctagtttatcaccaataatacttgacggaaggctcagtaattagttatgaatatggatatcctcaattcttaagacagcttgtaaatgtatttgtaaaaattgtatatatttttacagaaagtctatttccttgaaacgaaggaagtatcgaatttacattagtttttttcatacccttttgaactttgcaacttccgtaattaggaacctgtttcttacagcttttctatgctaaactttgttctgttcagttctagagtgtatacagaacgaattgatgtgtaactgtatgcagactggttgtagtggaacaaatctgataactatgcaggtttaaattttcttatctgattttggtaagtattccttagataggttttctttgaaaacctgggattgagaggttgatgaatggaaattctttcacttcattatatgcaagttttcaataattaggtctaagtggagttttaaggttactgatgacttacaaataatgggctctgattgggcaatactcatttgagttccttccatttgacctaatttaactggtgaaatttaaagtgaattcatgggctcatctttaaagcttttactaaaagattttcagctgaatggaactcattagctgtgtgcatataaaaagatcacatcaggtggatggagagacatttgatcccttgtttgcttaataaattataaaatgatggcttggaaaaaaaaaaaaaaaa

SEQ ID NO: 7 - DNA, 33 bp, artificial sequence
  Synthetic (DT564)
cgtgcgccaggctagctacaacgaatttcccag

SEQ ID NO: 8 - DNA, 33 bp, artificial sequence
  Synthetic (DT565)
tcccggttaggctagctacaacgacgtaccctg

SEQ ID NO: 9 - DNA, 33 bp, artificial sequence
  Synthetic (DT566)
tcatcactaggctagctacaacgactcccggtt

SEQ ID NO: 10 - DNA, 33 bp, artificial sequence
  Synthetic (DT567)
cgcatcccaggctagctacaacgatcgtagccc

SEQ ID NO: 11 - DNA, 33 bp, artificial sequence
  Synthetic (DT568)
tctcccgcaggctagctacaacgacccactcgt

SEQ ID NO: 12 - DNA, 33 bp, artificial sequence
  Synthetic (DT569)
gcgcccacaggctagctacaacgactcccgcat

SEQ ID NO: 13 - DNA, 33 bp, Artificial Sequence
  Synthetic (DT570)
cggcgcccaggctagctacaacgaatctcccgc

SEQ ID NO: 14 - DNA, 33 bp, Artificial Sequence
  Synthetic (DT571)
aggagaagaggctagctacaacgagcccggcgc

SEQ ID NO: 15 - DNA, 33 bp, Artificial Sequence
  Synthetic (DT572)
gcggctgtaggctagctacaacgaggggcgtgt

SEQ ID NO: 16 - DNA, 33 bp, Artificial Sequence
  Synthetic (DT573)
gtcccgggaggctagctacaacgagcggctgta

SEQ ID NO: 17 - DNA, 33 bp, Artificial Sequence
  Synthetic (DT574)
tcctggcgaggctagctacaacgacgggtcccg

SEQ ID NO: 18 - DNA, 33 bp, Artificial Sequence
  Synthetic (DT575)
caggtggcaggctagctacaacgacgggctgag

SEQ ID NO: 19 - DNA, 33 bp, Artificial Sequence
  Synthetic (DT576)
ggtggaccaggctagctacaacgaaggtggcac

SEQ ID NO: 20 - DNA, 33 bp, Artificial Sequence
  Synthetic (DT577)
gcctggacaggctagctacaacgactcggcgaa

SEQ ID NO: 21 - DNA, 33 bp, Artificial Sequence
  Synthetic (DT578)
ctgcctggaggctagctacaacgaatctcggcg

SEQ ID NO: 22 - DNA, 33 bp, Artificial Sequence
  Synthetic (DT579)
cctccaccaggctagctacaacgacgtggcaaa

SEQ ID NO: 23 - DNA, 33 bp, Artificial Sequence
  Synthetic (DT580)
gctcctccaggctagctacaacgacaccgtggc

SEQ ID NO: 24 - DNA, 33 bp, Artificial Sequence
  Synthetic (DT581)
cccagttcaggctagctacaacgacccgtccct

SEQ ID NO: 25 - DNA, 33 bp, Artificial Sequence
  Synthetic (DT582)
aggccacaaggctagctacaacgacctccccca

SEQ ID NO: 26 - DNA, 33 bp, Artificial Sequence
  Synthetic (DT583)
agaaggccaggctagctacaacgaaatcctccc

SEQ ID NO: 27 - DNA, 33 bp, Artificial Sequence
  Synthetic (DT584)
cacacatgaggctagctacaacgacccaccgaa

SEQ ID NO: 28 - DNA, 33 bp, Artificial Sequence
  Synthetic (DT585)
ccacacacaggctagctacaacgagaccccacc

SEQ ID NO: 29 - DNA, 33 bp, Artificial Sequence
  Synthetic (DT586)
ctccacacaggctagctacaacgaatgacccca

SEQ ID NO: 30 - DNA, 33 bp, Artificial Sequence
  Synthetic (DT587)
ctctccacaggctagctacaacgaacatgaccc

SEQ ID NO: 31 - DNA, 33 bp, Artificial Sequence
  Synthetic (DT588)
cccggttgaggctagctacaacgagctctccac

SEQ ID NO: 32 - DNA, 33 bp, Artificial Sequence
  Synthetic (DT589)
ggggcgacaggctagctacaacgactcccggtt

SEQ ID NO: 33 - DNA, 33 bp, Artificial Sequence
  Synthetic (DT590)
caggggcgaggctagctacaacgaatctcccgg

SEQ ID NO: 34 - DNA, 33 bp, Artificial Sequence
  Synthetic (DT591)
tgttgtccaggctagctacaacgacaggggcga

SEQ ID NO: 35 - DNA, 33 bp, Artificial Sequence
  Synthetic (DT592)
acagggcgaggctagctacaacgagttgtccac

SEQ ID NO: 36 - DNA, 33 bp, Artificial Sequence
  Synthetic (DT593)
agtcatccaggctagctacaacgaagggcgatg

SEQ ID NO: 37 - DNA, 33 bp, Artificial Sequence
  Synthetic (DT594)
actcagtcaggctagctacaacgaccacagggc

SEQ ID NO: 38 - DNA, 33 bp, Artificial Sequence
  Synthetic (DT595)
tatcctggaggctagctacaacgaccaggtgtg

SEQ ID NO: 39 - DNA, 33 bp, Artificial Sequence
  Synthetic (DT596)
cctccgttaggctagctacaacgacctggatcc

SEQ ID NO: 40 - DNA, 33 bp, Artificial Sequence
  Synthetic (DT597)
acaaaggcaggctagctacaacgacccagcctc

SEQ ID NO: 41 - DNA, 33 bp, Artificial Sequence
  Synthetic (DT598)
ggggccgtaggctagctacaacgaagttccaca

SEQ ID NO: 42 - DNA, 33 bp, Artificial Sequence
  Synthetic (DT599)
gaggccgcaggctagctacaacgagctggggcc

SEQ ID NO: 43 - DNA, 33 bp, Artificial Sequence
  Synthetic (DT600)
aagctcccaggctagctacaacgacagggccaa

SEQ ID NO: 44 - DNA, 33 bp, Artificial Sequence
  Synthetic (DT601)
ccagggtgaggctagctacaacgagcaagctcc

SEQ ID NO: 45 - DNA, 33 bp, Artificial Sequence
  Synthetic (DT602)
agataggcaggctagctacaacgaccagggtga

SEQ ID NO: 46 - DNA, 33 bp, Artificial Sequence
  Synthetic (DT603)
tggcccagaggctagctacaacgaaggcaccca

SEQ ID NO: 47 - DNA, 33 bp, Artificial Sequence
  Synthetic (DT604)
ttgacttcaggctagctacaacgattgtggccc

SEQ ID NO: 48 - DNA, 33 bp, Artificial Sequence
  Synthetic (DT605)
gggcaggcaggctagctacaacgagttgacttc

SEQ ID NO: 49 - DNA, 33 bp, Artificial Sequence
  Synthetic (DT606)
ggagccacaggctagctacaacgagaagcggtg

SEQ ID NO: 50 - DNA, 33 bp, Artificial Sequence
  Synthetic (DT607)
ccccaatgaggctagctacaacgacaggtcctt

SEQ ID NO: 51 - DNA, 33 bp, Artificial Sequence
  Synthetic (DT608)
agggaggcaggctagctacaacgaggacttccc

SEQ ID NO: 52 - DNA, 33 bp, Artificial Sequence
  Synthetic (DT609)
ttcctcccaggctagctacaacgacaggtatgc

SEQ ID NO: 53 - DNA, 33 bp, Artificial Sequence
  Synthetic (DT610)
tttttcccaggctagctacaacgacgctgtcct

SEQ ID NO: 54 - DNA, 33 bp, Artificial Sequence
  Synthetic (DT611)
gcggcctgaggctagctacaacgagctctgggt

SEQ ID NO: 55 - DNA, 33 bp, Artificial Sequence
  Synthetic (DT612)
ccctgttgaggctagctacaacgacatccctgg

SEQ ID NO: 56 - DNA, 33 bp, Artificial Sequence
  Synthetic (DT613)
tggctcccaggctagctacaacgagctccacgt

SEQ ID NO: 57 - DNA, 33 bp, Artificial Sequence
  Synthetic (DT614)
cacagccaaggctagctacaacgagtgccatgt

SEQ ID NO: 58 - DNA, 33 bp, Artificial Sequence
  Synthetic (DT615)
acccccataggctagctacaacgatccacacct

SEQ ID NO: 59 - DNA, 33 bp, Artificial Sequence
  Synthetic (DT616)
cagggcttaggctagctacaacgactcaccttc

SEQ ID NO: 60 - DNA, 33 bp, Artificial Sequence
  Synthetic (DT617)
gcccagggaggctagctacaacgagaggaaacc

SEQ ID NO: 61 - DNA, 33 bp, Artificial Sequence
  Synthetic (DT618)
tgctggtcaggctagctacaacgattgccatct

SEQ ID NO: 62 - DNA, 33 bp, Artificial Sequence
  Synthetic (DT673)
aagagttcaggctagctacaacgatcactacct

SEQ ID NO: 63 - DNA, 33 bp, Artificial Sequence
  Synthetic (DT674)
tttaccccaggctagctacaacgacccggaaga

SEQ ID NO: 64 - DNA, 33 bp, Artificial Sequence
  Synthetic (DT675)
cccagtttaggctagctacaacgacccatcccg

SEQ ID NO: 65 - DNA, 33 bp, Artificial Sequence
  Synthetic (DT676)
acaatgcgaggctagctacaacgacccagttta

SEQ ID NO: 66 - DNA, 33 bp, Artificial Sequence
  Synthetic (DT677)
aggccacaaggctagctacaacgagcgacccca

SEQ ID NO: 67 - DNA, 33 bp, Artificial Sequence
  Synthetic (DT678)
aaaaggccaggctagctacaacgaaatgcgacc

SEQ ID NO: 68 - DNA, 33 bp, Artificial Sequence
  Synthetic (DT679)
ttccacgcaggctagctacaacgaagtgccccg

SEQ ID NO: 69 - DNA, 33 bp, Artificial Sequence
  Synthetic (DT680)
cgctttccaggctagctacaacgagcacagtgc

SEQ ID NO: 70 - DNA, 33 bp, Artificial Sequence
  Synthetic (DT681)
ccttgtctaggctagctacaacgagctttccac

SEQ ID NO: 71 - DNA, 33 bp, Artificial Sequence
  Synthetic (DT682)
atacctgcaggctagctacaacgactccttgtc

SEQ ID NO: 72 - DNA, 33 bp, Artificial Sequence
  Synthetic (DT683)
tcaccaataggctagctacaacgactgcatctc

SEQ ID NO: 73 - DNA, 33 bp, Artificial Sequence
  Synthetic (DT684)
actcaccaaggctagctacaacgaacctgcatc

SEQ ID NO: 74 - DNA, 33 bp, Artificial Sequence
  Synthetic (DT685)
tccgactcaggctagctacaacgacaatacctg

SEQ ID NO: 75 - DNA, 33 bp, Artificial Sequence
  Synthetic (DT686)
gcgatccgaggctagctacaacgatcaccaata

SEQ ID NO: 76 - DNA, 33 bp, Artificial Sequence
  Synthetic (DT687)
aagctgcgaggctagctacaacgaccgactcac

SEQ ID NO: 77 - DNA, 33 bp, Artificial Sequence
  Synthetic (DT688)
aagtggccaggctagctacaacgaccaagctgc

SEQ ID NO: 78 - DNA, 33 bp, Artificial Sequence
  Synthetic (DT689)
aggtggtcaggctagctacaacgatcaggtaag

SEQ ID NO: 79 - DNA, 33 bp, Artificial Sequence
  Synthetic (DT690)
tctcctggaggctagctacaacgaccaaggctc

SEQ ID NO: 80 - DNA, 33 bp, Artificial Sequence
  Synthetic (DT691)
acaaaagtaggctagctacaacgacccagccgc

SEQ ID NO: 81 - DNA, 33 bp, Artificial Sequence
  Synthetic (DT692)
gtctggtcaggctagctacaacgattccgactg

SEQ ID NO: 82 - DNA, 33 bp, Artificial Sequence
  Synthetic (DT693)
tttttataaggctagctacaacgaagggatggg

SEQ ID NO: 83 - DNA, 33 bp, Artificial Sequence
  Synthetic (DT694)
acatttttaggctagctacaacgaaatagggat

SEQ ID NO: 84 - DNA, 33 bp, Artificial Sequence
  Synthetic (DT695)
tctgagacaggctagctacaacgattttataat

SEQ ID NO: 85 - DNA, 33 bp, Artificial Sequence
  Synthetic (DT696)
gctctgagaggctagctacaacgaatttttata

SEQ ID NO: 86 - DNA, 33 bp, Artificial Sequence
  Synthetic (DT697)
agtcaaccaggctagctacaacgacagctcccg

SEQ ID NO: 87 - DNA, 33 bp, Artificial Sequence
  Synthetic (DT698)
gtggctccaggctagctacaacgatcaccgcgg

(19) United States
(12) Patent Application Publication
(10) Pub. No.: US 2005/0064407 A1
(43) Pub. Date: Mar. 24, 2005

(54) Bcl-2 dnazymes

(76) Inventors: Lun-Quan Sun, Eastwood (AU); Li Wang, Eastwood (AU); Rachel Jane Turner, Glebe (AU); Edward George Saravolac, Windsor (CA); Crispin Rajnish Dass, Reservoir (AU)

Correspondence Address: BROWDY AND NEIMARK, P.L.L.C.;624 NINTH STREET, NW, SUITE 300, WASHINGTON, DC, 20001-5303, US

(21) Appl. No.: 10/479,832

(22) PCT Filed: Jun. 7, 2002
(86) PCT No.: PCT/AU02/00739

(30) Foreign Application Priority Data
Jun. 7, 2001 (AU) PR 5527

(51) Int. Cl. C12Q 1/68; C07H 21/04; C12N 9/22

(52) U.S. Cl. 435/6; 435/69.1; 435/199; 435/320.1; 435/325; 536/23.2

(57) ABSTRACT

The present invention provides DNAzymes which specifically cleaves mRNA transcribed from a member of the bcl-2 gene family selected from the group consisting of bcl-2, bcl-xl, bcl-w, bfl-1, brag-1, Mcl-1 and A1. The DNAzymes comprise (a) a catalytic domain that has the nucleotide sequence GGCTAGCTACAACGA (SEQ ID NO.1) and cleaves mRNA at any purine: pyrimidine cleavage site at which it is directed, (b) a binding domain contiguous with the 5′ end of the catalytic domain, and (c) another binding domain contiguous with the 3′ end of the catalytic domain. The binding domains are complementary to, and therefore hybridise with, the two regions immediately flanking the purine residue of the cleavage site within the bcl-2 gene family mRNA, at which DNAzyme-catalysed cleavage is desired. Each binding domain is at least six nucleotides in length, and both binding domains have a combined total length of at least 14 nucleotides.

### FIELD OF THE INVENTION

The present invention relates to DNAzymes targeted to bcl-2 gene family members and their use in cancer therapy. This invention further relates to use of these DNAzymes to treat and/or inhibit onset of human cancers. The DNAzymes accomplish this end by cleaving mRNA transcribed from members of the bcl-2 gene family thereby provoking apoptosis of cancer cells directly and/or increasing the sensitivity of cancer cells to chemotherapeutics.

### BACKGROUND OF THE INVENTION

Apoptosis and Bcl-2 Gene Family

Apoptosis is a complex process resulting in the regulated destruction of a cell, which plays a major role in normal development, cellular response to injury and carcinogenesis(Ellis et al., 1991). It has been suggested that an apoptotic component either contributes to, or accounts for, many human disease pathologies including cancer, viral infection and some neurological disorders (Ashkenazi and Dixit, 1998; Vocero-Akbani et al., 1999; Yakovlev et al., 1997).

The Bcl-2 family of proteins are among the most studied molecules in the apoptotic pathway. Bcl-2 gene was first identified in B-cell lymphomas where the causal genetic lesion has been characterised as a chromosomal translocation (t(14:18)) which places the Bcl-2 gene under the control of the immunoglobulin promoter. The resulting overexpression of Bcl-2 retards the normal course of apoptotic cell death that otherwise maintains B-cell homeostasis, resulting in B-cell accumulation and follicular lymphoma (Adams and Cory, 1998). This observation showed that cancers do not strictly arise from unrestrained cell proliferation, but could also be due to insufficient apoptotic turnover. In addition to follicular lymphomas, Bcl-2 levels are elevated in a broad range of other human cancers, indicating that this molecule may have a role in raising the apoptotic threshold in a broad spectrum of cancerous disorders.

The Bcl-2 gene family has at least 16 members involved in the apoptosis pathway. Some genes in this family are apoptosis inducers, including, bax, bak, bcl-Xs, bad, bid, bik and hrk, and others, such as bcl-2, bcl-XL, bcl-w, bfl-1, brag-1, Mcl-1 and A1 are apoptosis suppressors (Reed, 1998). Bcl-2 family members have been suggested to act through many different mechanisms, including pore formation in the outer mitochondrial membrane, through which cytochrome c(Cyt c) and other intermembrane proteins can escape; and heterodimerization between pro- and anti-apoptotic family members (Reed, 2000).

It has been suggested that a decrease in Bcl-2 levels or the inhibition of Bcl-2 activity might provoke apoptosis or at least sensitise cells to apoptotic death. In the absence of a clearly defined biochemical mechanism of action or activity for this family of cell-death regulatory proteins (for which conventional inhibitors could therefore be developed), gene therapy and antisense approaches have become a reasonable alternative. For example, an 18-mer all-phosphorothioate Bcl-2 antisense oligodeoxynucleotide (ODN), G-3139 that targets the first six codons of the human Bcl-2 open reading frame, has shown very promising results in both preclinical and clinical studies (Jansen et al., 1998; Waters et al., 2000). This antisense molecule binds to the Bcl-2 mRNA blocking translation of the mRNA into Bcl-2 protein and targeting the message for RNAse H-mediated degradation. The resultant decrease in bcl-2 levels in the treated cells alters the balance between pro-apoptotic and anti-apoptotic family members in favour of pro-apoptotic members resulting in apoptosis.

Using a similar strategy, antisense oligonucleotides to another member of the bcl-2 gene family bcl-xL has also been shown to be active in down-regulation of the bcl-xL expression, leading to an increased chemosensitivity in a range of cancer cells (Zangemeister-Wittke et al., 2000).

Catalytic DNA (DNAzyme)

In human gene therapy, antisense nucleic acid technology has been one of the major tools of choice to inactivate genes whose expression causes disease and is thus undesirable. The anti-sense approach employs a nucleic acid molecule that is complementary to, and thereby hybridizes with, a mRNA molecule encoding an undesirable gene. Such hybridization leads to the inhibition of gene expression.

Anti-sense technology suffers from certain drawbacks. Anti-sense hybridization results in the formation of a DNA/target mRNA heteroduplex. This heteroduplex serves as a substrate for RNAse H-mediated degradation of the target mRNA component. Here, the DNA anti-sense molecule serves in a passive manner, in that it merely facilitates the required cleavage by endogenous RNAse H enzyme. This dependence on RNAse H confers limitations on the design of anti-sense molecules regarding their chemistry and ability to form stable heteroduplexes with their target mRNA's. Anti-sense DNA molecules also suffer from problems associated with non-specific activity and, at higher concentrations, even toxicity.

As an alternative to anti-sense molecules, catalytic nucleic acid molecules have shown promise as therapeutic agents for suppressing gene expression, and are widely discussed in the literature (Haseloff and Gerlach 1988; Breaker 1994; Koizumi et al 1993; Kashani-Sabet et al 1992; Raillard et al 1996; and Carmi et al 1998) Thus, unlike a conventional anti-sense molecule, a catalytic nucleic acid molecule functions by actually cleaving its target mRNA molecule instead of merely binding to it. Catalytic nucleic acid molecules can only cleave a target nucleic acid sequence if that target sequence meets certain minimum requirements. The target sequence must be complementary to the hybridizing regions of the catalytic nucleic acid, and the target must contain a specific sequence at the site of cleavage.

Catalytic RNA molecules (“ribozymes”) are well documented (Haseloff and Gerlach 1988; Symonds 1994; and Sun et al 1997), and have been shown to be capable of cleaving both RNA (Haseloff and Gerlach 1988) and DNA (Raillard et al 1996) molecules. Indeed, the development of in vitro selection and evolution techniques has made it possible to obtain novel ribozymes against a known substrate, using either random variants of a known ribozyme or random-sequence RNA as a starting point (Pan 1997; Tsang and Joyce 1996; and Breaker 1994).

Ribozymes, however, are highly susceptible to enzymatic hydrolysis within the cells where they are intended to perform their function. This in turn limits their pharmaceutical applications.

Recently, a new class of catalytic molecules called “DNAzymes” was created (Breaker and Joyce 1995; Santoro and Joyce 1997). DNAzymes are single-stranded, and cleave both RNA (Breaker (1994; Santoro and Joyce 1997) and DNA (Carmi et al 1998). A general model for the DNAzyme has been proposed, and is known as the “10-23” model. DNAzymes following the “10-23” model, also referred to simply as “10-23 DNAzymes”, have a catalytic domain of 15 deoxyribonucleotides, flanked by two substrate-recognition domains of seven to nine deoxyribonucleotides each. In vitro analyses show that this type of DNAzyme can effectively cleave its substrate RNA at purine:pyrimidine junctions under physiological conditions (Santoro and Joyce 1998).

Several groups have examined the activity of DNAzymes in biological systems. DNAzyme molecules targeting c-myc were found to suppress SMC proliferation after serum stimulation (Sun et al 1997). Two studies have explored the activity and specificity of DNAzymes targeting the bcr-abI fusion in Philadelphia chromosome positive leukemia cells; Wu et al., 1999). The activity of these DNAzymes compared favourably with previous work with hammerhead ribozymes and antisense oligonucleotides (Gewirtz et al., 1998).

More recently a 10-23 DNAzyme targeting the transcription factor Egr-1 has been shown to inhibit smooth muscle cell proliferation in cell culture and neointima formation in the rat carotid artery damaged by ligation injury or balloon angioplasty. (Santiago et al., 1999). Suppression of Egr-1 was also monitored at the RNA and protein level in treated smooth muscle cells by northern and western blot analysis respectively. This was the first evidence of DNAzyme efficacy in vivo, and furthermore the activity displayed by this anti-Egr-1 molecule could potentially find application in various forms of cardiovascular disease such as restenosis.

### SUMMARY OF THE INVENTION

The present inventors have determined that the level of expression of bcl-2 gene family members can be inhibited by DNAzymes.

Accordingly in a first aspect the present invention consists in a DNAzyme which specifically cleaves mRNA transcribed from a member of the bcl-2 gene family, the DNAzyme comprising (a) a catalytic domain that has the nucleotide sequence GGCTAGCTACAACGA (SEQ ID No.1) and cleaves mRNA at any purine:pyrimidine cleavage site at which it is directed, (b) a binding domain contiguous with the 5′ end of the catalytic domain, and (c) another binding domain contiguous with the 3′ end of the catalytic domain, wherein the binding domains are complementary to, and therefore hybridise with, the two regions immediately flanking the purine residue of the cleavage site within the bcl-2 gene family mRNA, at which DNAzyme-catalysed cleavage is desired, and wherein each binding domain is at least six nucleotides in length, and both binding domains have a combined total length of at least 14 nucleotides.

This invention also provides a method to enhance the sensitivity of malignant or virus infected cells to therapy by modulating expression level of a member of the bcl-2 gene family using catalytic DNA.

It is preferred that the bcl-2 gene family member is selected from the group consisting of bcl-2, bcl-xl, bcl-w, bfl-1, brag-1, Mcl-1 and A1. It is particularly preferred that the bcl-2 gene family member is bcl-2 or bcl-xl.

### BRIEF DESCRIPTION OF THE FIGURES

FIG. 1: “10-23” DNAzyme (PO-DNAzyme) and its phosphorothioate modified version (PS-DNAzyme). Panel A contain illustration for 10-23 DNAzyme. Watson-Crick interactions for DNAzyme-substrate complex is represented by generic ribonucleotides (N) in the target (top) and the corresponding DNAzyme (N) in the arms of the DNAzyme (bottom). The defined sequence in the loop joining the arms and spanning a single unpaired purine at the RNA target site of the model represents the conserved catalytic motif. Panel B shows a chemically modified version of the DNAzyme. * represents a phosphorothioate linkage.

FIG. 2: Stability of phosphorothioate-modified DNAzyme oligonucleotides in human serum. DNAzymes with 1,3, or 5 phosphorothioate linkages at each arm were incubated with fresh human serum and sampled at various time points. From each sample, intact oligonucleotides were extracted by phenol and ³²P-labelled using polynucleotide kinase. The labelled reactions were subjected to a gel electrophoresis. Percentage of intact oligos is calculated from: intensity at various time points/intensity at 0 time point×100, as measured by PhosphoImage.

FIG. 3: TMP-mediated DNAzyme transfection of PC3 cells. 2 μM FITC-labelled DNAzyme was complexed with TMP at a charge ratio of 0,1,3,5,10 and 20. The result from FACS analysis are represented.

FIG. 4: Chemosensitization of PC3 cells by Bcl-xL DNAzyme. PC3 cells were treated with DNAzyme/TMP complex for 4 hours. The medium was then replaced with fresh DMEM containing 10% FBS and 5 μM Carboplatin and further incubated for 72 hours. MTS assays were performed for cell proliferation of all the samples. % cell death is derived from the percentage of OD490 from the Carboplatin-treated samples of that from untreated PC3 cells.

FIG. 5: Chemosensitization of PC3 tumour cells in human xenograph mouse model (PC3) by anti-bcl-xL. Nude mice bearing established, subcutaneously growing PC3 tumour xenograft either remained untreated (saline) or were treated with DNAzyme oligo, Taxol or DNAzyme+Taxol. DNAzyme DT882 was delivered using an osmotic pump and Taxol was administrated via i.p. route weekly. Tumour size was measured at the time points indicated.

FIG. 6: Chemosensitization of MDA-MB231 human xenograph breast cancer mouse model by anti-bcl-xL DNAzyme. Nude mice bearing established, subcutaneously growing MDA-MB231 tumour xenograft either remained untreated (saline) or were treated with DNAzyme oligo, Taxol or DNAzyme+Taxol. DNAzyme DT882 was delivered using an osmotic pump and Taxol was administrated via i.p. route weekly. Tumour size was measured at the time points indicated.

FIG. 7: Chemosensitization of MDA-MB231 human xenograph breast cancer mouse model by anti-bcl-2 DNAzyme. Nude mice bearing established, subcutaneously growing MDA-MB231 tumour xenograft either remained untreated (saline) or were treated with DNAzyme oligo, Taxol or DNAzyme+Taxol. DNAzyme DT912 was delivered using an osmotic pump and Taxol was administrated via i.p. route weekly. Tumour size was measured at the time points indicated.

FIG. 8: Western analysis of Bcl-2 expression level I in MDA-MB 231 tumors. Bcl-2 expression levels were determined by densitometry analysis of western blots of protein extracts of tumors removed from groups of 6 mice after15 days of treatment. The relative bcl-2 expression was calculated based on the ratio of Bcl-2 to β-actin levels.

FIG. 9: Chemosensitization of human prostate tumour cells in xenograph mouse model by anti-bcl-2 DNAzyme. Nude mice bearing established, subcutaneously growing PC3 tumour xenograft either remained untreated (saline) or were treated with DNAzyme oligo, Taxol or DNAzyme+Taxol. DNAzyme DT912 was delivered using an osmotic pump and Taxol was administrated via i.p. route weekly. Tumour size was measured at the time points indicated.

FIG. 10: Chemosensitization of human melanoma tumour cells in human xenograph mouse model (518A2) by anti-bcl-2 DNAzyme SCID mice bearing established, subcutaneously growing 518A2 tumour xenograft either remained untreated (saline) or were treated with DNAzyme oligo, DTIC or DNAzyme+DTIC. DNAzyme DT912 was delivered using an osmotic pump and DTIC was administrated via i.p. route weekly. Tumour size was measured at the time points indicated and the fold of tumor growth was plotted in the figure.

### DETAILED DESCRIPTION OF THE INVENTION

In a first aspect the present invention consists in a DNAzyme which specifically cleaves mRNA transcribed from a member of the bcl-2 gene family selected from the group consisting of bcl-2, bcl-xl, bcl-w, bfl-1, brag-1, Mcl-1 and A1, the DNAzyme comprising (a) a catalytic domain that has the nucleotide sequence GGCTAGCTACAACGA (SEQ ID NO.1) and cleaves mRNA at any purine:pyrimidine cleavage site at which it is directed, (b) a binding domain contiguous with the 5′ end of catalytic domain, wherein the binding domains are complementary to, and therefore hybridise with, the two regions immediately flanking the purine residue of the cleavage site within the bcl-2 gene family mRNA, at which DNAzyme-catalysed cleavage is desired, and wherein each binding domain is at least six nucleotides in length, and both binding domains have a combined total length of at least 14 nucleotides.

This invention also provides a method to enhance the sensitivity of malignant or virus infected cells to therapy by modulating expression level of a member of the bcl-2 gene family selected from the group consisting of bcl-2, bcl-xl, bcl-w, bfl-1, brag-1, Mcl-1 and A1 using catalytic DNA (see Table 6).

In a preferred embodiment the DNAzyme is 29 to 39 nucleotides in length.

It is preferred that the bcl-2 gene family member is bcl-2 or bcl-xl. Where the bcl-2 gene family member is bcl-2 it is preferred that the DNAzyme is selected from those set out in Table 1. Where the bcl-2 gene family member is bcl-xl it is preferred that the DNAzyme is selected from those set out in Table 2.

Where the DNAzyme cleaves bcl-2 mRNA it is further preferred that the DNAzyme cleaves bcl-2 mRNA at position 455, 729, 1432, 1806 or 2093 (SEQ ID NO.2). It is particularly preferred that the sequence of the DNAzyme is as set out in SEQ ID NO 24, 45, 53, 55 or 57.

Where the DNAzyme cleaves bcl-xl mRNA it is further preferred that the DNAzyme cleaves bcl-xl mRNA at position 126, 129 or 135 (SEQ ID NO.3). It is particularly preferred that the sequence of the DNAzyme is as set out in SEQ ID NO 82, 83 or 84.

The present invention comprehends DNAzyme compounds capable of modulating expression bcl-2 gene family members, in particular human bcl-2 and bcl-xL genes. These genes inhibit apoptosis and therefore inhibitors of these genes, particularly specific inhibitors of bcl-2 and bcl-xL such as the DNAzyme compounds of the present invention are desired as promoters of apoptosis.

More specifically, this application provides a set of DNAzymes which specifically cleaves mRNA of the bcl-2 and bcl-xL genes, comprising (a) a catalytic domain that has the nucleotide sequence GGCTAGCTACAACGA (SEQ ID NO.1) and cleaves mRNA at any purine:pyrimidine cleavage site at which it is directed, (b) a binding domain contiguous with the 5′ end of the catalytic domain, and (c) another binding domain contiguous with the 3′ end of the catalytic domain, wherein the binding-domains are complementary to, and therefore hybridise with, the two regions immediately flanking the purine residue of the cleavage site within the mRNA of the bcl-2 and bcl-xL genes, respectively, at which DNAzyme-catalysed cleavage is desired, and wherein each binding domain is at least six nucleotides in length, and both binding domains have a combined total length of at least 14 nucleotides.

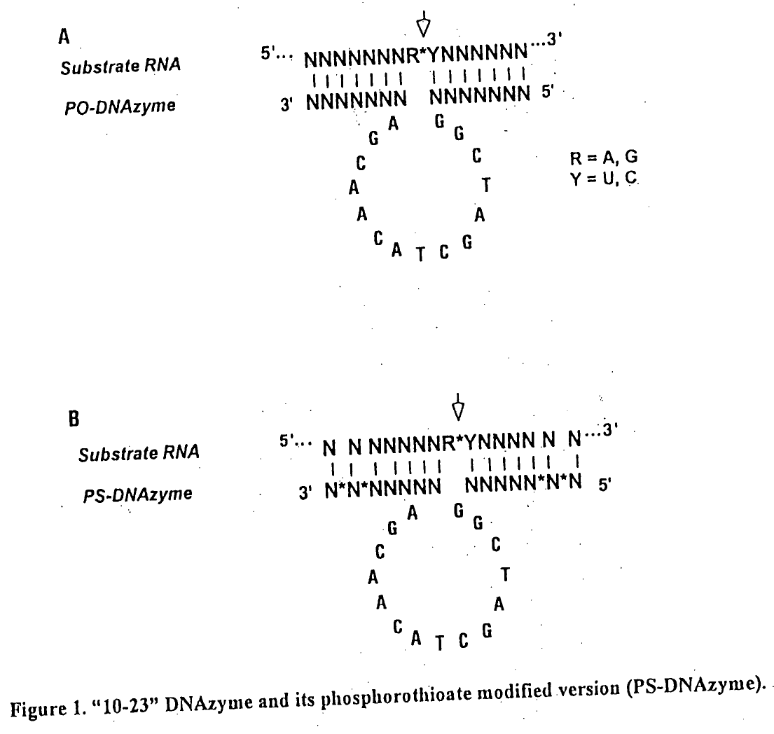

As used herein, “DNAzyme” means a DNA molecule that specifically recognizes a distinct target nucleic acid sequence, which can be either pre-mRNA or mRNA transcribed from the target genes. The instant DNAzyme cleaves RNA molecules, and is of the “10-23” model, as shown in FIG. 1, named so for historical reasons. This type of DNAzyme is described in Santoro et al 1997. The RNA target sequence requirement for the 10-23 DNAzyme is any RNA sequence consisting of NNNNNNNR*YNNNNNN, NNNNNNNR*YNNNNN or NNNNNNR*YNNNNNNN, where R*Y is the cleavage site, R is A or G, Y is U or C and N is any of G, U, C, or A.

Within the parameters of this invention, the binding domain lengths (also referred to herein as “arm lengths”) can be any permutation, and can be the same or different. In the preferred embodiment, each binding domain is nine nucleotides in length.

In this invention, any contiguous-purine:pyrimidine nucleotide pair within mRNA transcribed from a member of the bcl-2 gene family selected from the group consisting of bcl-2, bcl-xL, bcl-w, bfl-1, brag-1, Mcl-1 and A1 can serve as a cleavage site. In the preferred embodiment, purine:uracil is the purine:pyrimidine cleavage site.

As used herein the term “specifically cleaves” refers to a DNAzyme which cleaves mRNA, particularly in vivo, transcribed from the specified gene such that the activity of the gene is modulated.

Targeting a DNAzyme compound to a particular nucleic acid is generally a multistep process. The process usually begins with the identification of a nucleic acid sequence whose function is to be modulated. This may be, for example, cellular gene (or mRNA transcribed from the gene) whose expression is associated with a particular disorder or disease state, or a nucleic acid molecule from an infectious agent. In the present invention, the preferred targets are members of the bcl-2 gene family, in particular the nucleic acids encoding bcl-2 and bcl-xL. The targeting process also includes determination of sites within these genes for the DNAzyme catalytic activity to occur such that the desired effect, eg., detection or modulation of the proteins, will result. Within the context of the present invention, the preferred target sites are determined by a multiplex in vitro selection method and cell-based screening assays.

In applying DNAzyme-based treatments, it is important that the DNAzymes be as stable as possible against degradation in the intracellular milieu. One means of accomplishing this is by phosphorothioate modifications at both ends of the DNAzymes. Accordingly, in the preferred embodiment, two phosphorothioate linkages are introduced into both the 5′ and 3′ ends of the DNAzymes. In addition to phosphorothioate modification, the DNAzymes can contain other modifications. These include, for example, the 3′-3′ inversion at the 3′ end, N3′-P5′ phosphoramidate linkages, peptide-nucleic acid linkages, and 2′-O-methyl. These are well known in the art (Wagner 1995).

The DNAzymes of the present invention can be utilised for diagnostics, therapeutics, and prophylaxis and as research reagents and kits. For therapeutics, an animal, preferable a human, suspected of having a disease or disorder which can be treated by modulation the expression of a member of the bcl-2 gene family, in particular bcl-2 and bcl-xL, is treated by administering DNAzyme compounds in accordance with this invention.

The DNAzyme compounds of this invention are useful for research and diagnostics, because these compounds hybridise to and cleave nucleic acids encoding bcl-2 and bcl-xL, enabling the assays to be easily constructed to exploit this fact. The means for the detection include, for example, conjugation of a flourophore and a quencher to the substrate of the DNAzymes.

The present invention also includes pharmaceutical compositions and formulations, which comprise the DNAzyme compounds of the invention. The pharmaceutical compositions of the present invention may be administered in a number of ways depending upon whether local or systemic treatment is desired and upon the area to be treated. The administration can be topical, pulmonary, oral or parenteral.

Pharmaceutical compositions and formulations for topical administration may include transdermal patches, ointments, lotions, creams, gels, drops, suppositories, sprays, liquids and powders. Conventional pharmaceutical carriers, aqueous, powders or oily bases, thickeners and the like may be necessary or desirable.

Composition and formulations for oral administration include powders or granules, suspensions or solutions in water or non-aqueous media, capsules satchels or tablets.

The DNAzymes of the present invention can be used to increase the susceptibility of tumour cells to anti-tumour therapies such as chemotherapy and radiation therapy.

Accordingly in certain embodiments of this invention there are provided liposomes and other compositions containing (a) one or more DNAzyme compounds of the invention and (b) one or more chemotherapeutic agents which function by a non-hybridisation mechanism. Examples of such chemotherapeutic agents include, but are not limited to, anticancer drugs such as taxol, daunorubicin, dacitinomycin, doxorubicin, bleomycin, mitomycin, nitrogen mustard, chlorambucil, melphalan, cyclophosphamide, 6-mercaptopurine, 6-thioguanine, cytarabine, 5-flurouracil, floxuridine, methotrexate, colchicine, vincristine, vinlastin, etoposide, cisplatin. See, generally, The Merck Manual of Diagnosis and Therapy, 15th Ed., Berkow et al eds., 1987, Rahway, N.J., pp 1206-1228.

The formulation of the therapeutic compositions and their subsequent administration is believed to be within the skill of those in the art. Dosing is dependent on severity and responsiveness of the disease state to be treated, with the course of treatment lasting from several days to several months, or until a cure is effected or diminution of the disease state is achieved. Optimal dosing schedules can be determined from measurements of drug accumulation in the body of the patient. Persons of ordinary skill can easily determine optimum dosages, dosing methodologies and repetition rates. In general, dosage is from 0.01 μg to 100 g per kg of body weight and may be given daily, weekly, monthly or yearly.

In a further aspect the present invention consists in a method of treating tumours in a subject, the method comprising administering to the subject a composition comprising the DNAzyme of the first aspect of the present invention.

In a preferred embodiment the composition further comprises a chemotherapeutic agent.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in the specification are herein incorporated by reference.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia before the priority date of each claim of this application.

In order that the nature of the present invention may be more clearly understood preferred forms thereof will be described with reference to the following Examples.

### EXAMPLES

### Example 1

Identification of Cleavable sites in the bcl-2 and bcl-xL mRNA.

Two genes, Bcl-2 and Bcl-XL were chosen as DNAzyme targets for the treatment of cancers. These genes both belong to the Bcl-2 family and both are apoptosis repressors. Their products are found in elevated levels in many cancer types including malignant melanoma, ovarian cancer, lymphoma and prostate cancer.

Identification of Cleavage sites in bcl-2 mRNA for DNAzyme Design:

A partial bcl-2 cDNA clone was generated from cellular RNA, which contained 31 bp of the 5′ UTR, 720 bp of ORF and 2.2 kb of the 3′ UTR sequences. By scanning the mRNA corresponding to the bcl-2 clone, 210 potential AU and GU cleavage sites were identified and these sites were further subjected to two thermodynamic analyses. The first analysis was on the thermodynamic stability of the enzyme-substrate heteroduplex as predicted by the hybridisation free energy (Sugimoto et al., 1995, Cairns et al., 1999). DNA enzymes with the greatest heteroduplex stability indicated by a low free energy of hybridisation (calculated using the nearest neighbour method), was often found to have the greatest kinetic activity. The selection parameters included a cut-off value of −ΔG° kcal/mol of less than 25. The second analysis was to examine if the arms of the DNAzyme had a high hairpin melting temperature (Tm), thus to avoid any intramolecular bonds (Cairns et al., 1999; Santoro & Joyce 1998). After completion of these analyses, 55 (fifty-five) DNAzymes were designed and synthesised for in vitro multiplex selection. The sequence of these DNAzymes is set out in Table 1.

TABLE 1

Summary list of bcl-2 DNAzymes.

| | | | |
|---|---|---|---|
| SEQ. | −ΔG ° | Activity | Activity |

| | | | | | |
|---|---|---|---|---|---|
| Un- | ID | 2/2 | kcal/ | In | In |

| | | | | | | |
|---|---|---|---|---|---|---|
| modified¹ | NO | PS³ | Sequence | mol | vitro² | Cells⁴ |

| | | | | | | |
|---|---|---|---|---|---|---|
| DT564 | 7 | | cgtgcgccaggctagctacaacgaatttcccag | 28.8 | | |
| DT565 | 8 | | tcccggttaggctagctacaacgacgtaccctg | 28.6 | | |
| DT566 | 9 | DT891 | tcatcactaggctagctacaacgactcccggtt | 26 | + | NO |
| DT567 | 10 | | cgcatcccaggctagctacaacgatcgtagccc | 30.7 | | |
| DT568 | 11 | | tctcccgcaggctagctacaacgacccactcgt | 31.6 | | |
| DT569 | 12 | | gcgcccacaggctagctacaacgactcccgcat | 33 | | |
| DT570 | 13 | | cggcgcccaggctagctacaacgaatctcccgc | 33.8 | | |
| DT571 | 14 | | aggagaagaggctagctacaacgagcccggcgc | 28.9 | | |
| DT572 | 15 | | gcggctgtaggctagctacaacgaggggcgtgt | 30.4 | | |
| DT573 | 16 | | gtcccgggaggctagctacaacgagcggctgta | 31.2 | | |
| DT574 | 17 | DT892 | tcctggcgaggctagctacaacgacgggtcccg | 31.7 | +++ | NO |
| DT575 | 18 | DT893 | caggtggcaggctagctacaacgacgggctgag | 27.7 | +++ | NO |
| DT576 | 19 | DT894 | ggtggaccaggctagctacaacgaaggtggcac | 27.8 | +++ | NO |
| DT577 | 20 | | gcctggacaggctagctacaacgactcggcgaa | 27.6 | | |
| DT578 | 21 | | ctgcctggaggctagctacaacgaatctcggcg | 28.1 | | |
| DT579 | 22 | | cctccaccaggctagctacaacgacgtggcaaa | 27.6 | | |
| DT580 | 23 | | gctcctccaggctagctacaacgacaccgtggc | 31.4 | | |
| DT581 | 24 | DT895 | cccagttcaggctagctacaacgacccgtccct | 32.8 | + | YES |
| DT582 | 25 | | aggccacaaggctagctacaacgacctccccca | 32.3 | | |
| DT583 | 26 | | agaaggccaggctagctacaacgaaatcctccc | 27 | | |
| DT584 | 27 | | cacacatgaggctagctacaacgacccaccgaa | 25.1 | | |
| DT585 | 28 | | ccacacacaggctagctacaacgagaccccacc | 29.5 | | |
| DT586 | 29 | | ctccacacaggctagctacaacgaatgacccca | 27.7 | | |
| DT587 | 30 | | ctctccacaggctagctacaacgaacatgaccc | 26.3 | | |
| DT588 | 31 | | cccggttgaggctagctacaacgagctctccac | 29.6 | | |
| DT589 | 32 | | ggggcgacaggctagctacaacgactcccggtt | 30.7 | | |
| DT590 | 33 | | caggggcgaggctagctacaacgaatctcccgg | 29.6 | | |
| DT591 | 34 | | tgttgtccaggctagctacaacgacaggggcga | 27.5 | | |
| DT592 | 35 | | acagggcgaggctagctacaacgagttgtccac | 26.7 | | |
| DT593 | 36 | DT896 | agtcatccaggctagctacaacgaagggcgatg | 25.4 | + | NO |
| DT594 | 37 | DT897 | actcagtcaggctagctacaacgaccacagggc | 26.7 | + | NO |
| DT595 | 38 | | tatcctggaggctagctacaacgaccaggtgtg | 25.5 | | |
| DT596 | 39 | DT898 | cctccgttaggctagctacaacgacctggatcc | 28.6 | + | NO |
| DT597 | 40 | | acaaaggcaggctagctacaacgacccagcctc | 27.4 | | |
| DT598 | 41 | DT899 | ggggccgtaggctagctacaacgaagttccaca | 28.8 | + | YES |
| DT599 | 42 | | gaggccgcaggctagctacaacgagctggggcc | 33.4 | | |
| DT600 | 43 | DT900 | aagctcccaggctagctacaacgacagggccaa | 28.4 | + | YES |
| DT6O1 | 44 | DT901 | ccagggtgaggctagctacaacgagcaagctcc | 28.2 | + | NO |
| DT602 | 45 | DT902 | agataggcaggctagctacaacgaccagggtga | 25.3 | + | YES |
| DT603 | 46 | DT903 | tggcccagaggctagctacaacgaaggcaccca | 31.3 | + | NO |
| DT604 | 47 | DT904 | ttgacttcaggctagctacaacgattgtggccc | 25.7 | + | NO |
| DT605 | 48 | DT905 | gggcaggcaggctagctacaacgagttgacttc | 26.5 | +++ | YES |
| DT606 | 49 | | ggagccacaggctagctacaacgagaagcggtg | 26.5 | | |
| DT607 | 50 | DT906 | ccccaatgaggctagctacaacgacaggtcctt | 27.5 | +++ | NO |
| DT608 | 51 | | agggaggcaggctagctacaacgaggacttccc | 28.5 | | |
| DT609 | 52 | DT907 | ttcctcccaggctagctacaacgacaggtatgc | 27.5 | +++ | NO |
| DT610 | 53 | DT908 | tttttcccaggctagctacaacgacgctgtcct | 27.9 | + | YES |
| DT611 | 54 | DT909 | gcggcctgaggctagctacaacgagctctgggt | 31.1 | +++ | NO |
| DT612 | 55 | DT910 | ccctgttgaggctagctacaacgacatccctgg | 28.4 | +++ | YES |
| DT613 | 56 | DT911 | tggctcccaggctagctacaacgagctccacgt | 31.4 | +++ | NO |
| DT614 | 57 | DT912 | cacagccaaggctagctacaacgagtgccatgt | 26.7 | +++ | YES |
| DT615 | 58 | DT913 | acccccataggctagctacaacgatccacacct | 30.1 | +++ | NO |
| DT616 | 59 | DT914 | cagggcttaggctagctacaacgactcaccttc | 25.6 | +++ | NO |
| DT617 | 60 | DT915 | gcccagggaggctagctacaacgagaggaaacc | 27.1 | +++ | NO |
| DT618 | 61 | DT916 | tgctggtcaggctagctacaacgattgccatct | 26.5 | +++ | NO |

2. In the process of in vitro selection, twenty-six active DNAzymes were identified (26/55).
3. The in vitro selected DNAzymes were further chemically modified using two phosphorothioate linkages at both ends and renamed as indicated (Wagner 1995).
4. The modified DNAzymes were subjected to cell-based assay in which the bcl-2 protein level was measured by Western blots. Eight DNAzymes were shown active in down-regulation of Bcl-2 protein.
   Identification of Cleavage Sites in bcl-xL mRNA for DNAzyme Design:

As for the bcl-2 DNAzyme selection, total of 26 DNAzymes were designed and synthesised for the bcl-xL mRNA, based on the sequence scanning, and −Δ° G/Tm analyses. The sequence of these DNAzymes is set out in Table 2.

TABLE 2

Summary list of bcl-xL DNAzymes.

| | | | |
|---|---|---|---|
| SEQ. | −Δ ° G | In | Activity |

| | | | | | |
|---|---|---|---|---|---|
| Un- | ID | 2/2 | kcal/ | vitro | In |

| | | | | | | |
|---|---|---|---|---|---|---|
| modified¹ | NO | PS³ | Sequence | mol | Activity² | cells⁴ |

| | | | | | | |
|---|---|---|---|---|---|---|
| DT673 | 62 | DT861 | aagagttcaggctagctacaacgatcactacct | 21.70 | + | |
| DT674 | 63 | DT862 | ccccaggctagctacaacgacccggaaga | 27.70 | + | |
| DT675 | 64 | DT863 | ccagtttaggctagctacaacgacccatcccg | 30.10 | + | |
| DT676 | 65 | DT864 | caatgcgaggctagctacaacgacccagttta | 23.60 | | |
| DT677 | 66 | DT865 | aggccacaaggctagctacaacgagcgacccca | 30.40 | | |
| DT678 | 67 | DT866 | aaaaggccaggctagctacaacgaaatgcgacc | 22.70 | | |
| DT679 | 68 | DT867 | ccacgcaggctagctacaacgaagtgccccg | 30.90 | | |
| DT680 | 69 | DT868 | gctttccaggctagctacaacgagcacagtgc | 27.60 | + | |
| DT681 | 70 | DT869 | ccttgtctaggctagctacaacgagctttccac | 25.80 | ++ | |
| DT682 | 71 | DT870 | tacctgcaggctagctacaacgactccttgtc | 25.60 | +++ | |
| DT683 | 72 | DT871 | tcaccaataggctagctacaacgactgcatctc | 22.50 | ++ | |
| DT684 | 73 | DT872 | actcaccaaggctagctacaacgaacctgcatc | 24.80 | | |
| DT685 | 74 | DT873 | ccgactcaggctagctacaacgacaatacctg | 23.20 | | |
| DT686 | 75 | DT874 | cgatccgaggctagctacaacgatcaccaata | 24.40 | + | |
| DT687 | 76 | DT875 | agctgcgaggctagctacaacgaccgactcac | 25.40 | + | |
| DT688 | 77 | DT876 | agtggccaggctagctacaacgaccaagctgc | 27.10 | ++ | ++ |
| DT689 | 78 | DT877 | aggtggtcaggctagctacaacgatcaggtaag | 22.80 | ++ | ++ |
| DT690 | 79 | DT879 | ctcctggaggctagctacaacgaccaaggctc | 27.10 | +++ | |
| DT691 | 80 | DT880 | acaaaagtaggctagctacaacgacccagccgc | 25.20 | | |
| DT692 | 81 | DT881 | tctggtcaggctagctacaacgattccgactg | 25.40 | +++ | + |
| DT693 | 82 | DT882 | tttataaggctag ctacaacgaagggatggg | 18.90 | + | +++ |
| DT694 | 83 | DT883 | acatttttaggctagctacaacgaaatagggat | 17.40 | + | + |
| DT695 | 84 | DT884 | ctgagacaggctagctacaacgattttataat | 16.80 | + | + |
| DT696 | 85 | DT885 | ctctgagaggctagctacaacgaatttttata | 19.40 | +++ | |
| DT697 | 86 | DT886 | gtcaaccaggctagctacaacgacagctcccg | 27.50 | ++ | |
| DT698 | 87 | DT887 | tggctccaggctagctacaacgatcaccgcgg | 30.50 | | |

1. After screening the 926-bp bcl-xL cDNA clone, 81 potential cleavable AU or GU sites were found and these sites were subjected to thermodynamic analyses. Based on the threshold of −25 kcal/mol as selection criteria, twenty-six DNAzymes were synthesized for in vitro cleavage selection (26/81).
2. In the process of in vitro selection, eighteen active DNAzymes were identified (18/26).
3. All twenty-six DNAzymes were further chemically modified using two phosphorothioate linkages at both ends and renamed as indicated.
4. The modified DNAzymes were subjected to cell-based assay in which the bcl-xL protein level was measured by Western blots. Six DNAzymes were shown active in down-regulation of Bcl-xL protein (6/26).

### Example 2

Multiplex Selection of Active DNAzymes In Vitro:

In order to efficiently select active DNAzymes, in vitro selection was performed using a multiplex method, which enables a pool of DNAzymes to be screened for their ability to access and cleave RNA substrate under simulated physiological conditions (Cairns et al., 1999). The DNAzymes (0 nM, 5 nM, 50 nM and 500 nM) and RNA substrate (400 nM) were pre-equilibrated separately for 10 min at 37° C. in equal volumes of 50 mM Tris-HCL, pH 7.5 10 mM MgCl2, 150 mM NaCl and 0.01% SDS. Reaction was initiated by mixing the DNAzymes and substrate together. After 1 hr the reaction was stopped by extraction in 100 μl phenol/chloroform and recovered by ethanol precipitation.

The primers for bcl-2 cleavage detection are:

| | |
|---|---|
| 5′-cacagcattaaacattgaacag-3′ | (SEQ ID NO. 90) |
| 5′-tggaactttttttttgtcagg-3′ | (SEQ ID NO. 91) |
| 5′-tcctcacgttcccagccttc-3′ | (SEQ ID NO. 92) |
| 5′-cagacattcggagaccacac-3′ | (SEQ ID NO. 93) |
| 5′-cagtattgggagttgggggg-3′ | (SEQ ID NO. 94) |
| 5′-ccaactcttttcctcccacc-3′ | (SEQ ID NO. 95) |
| 5′-cgacgttttgcctgaagactg-3′ | (SEQ ID NO. 96) |
| 5′-cagggccaaactgagcagag-3′ | (SEQ ID NO. 97) |
| 5′-atcctcccccagttcacccc-3′ | (SEQ ID NO. 98) |
| 5′-ggatgcggctgtatgggg-3′; | (SEQ ID NO. 99) and |
| 5′-aggccacgtaaagcaactctc-3′. | (SEQ ID NO. 100) |

The primers for bcl-xL cleavage detection are:

| | |
|---|---|
| 5′-cgggttctcctggtggca-3′ | (SEQ ID NO. 101) |
| 5′-cctttcggctctcggctg-3′ | (SEQ ID NO. 102) |
| 5′-ccgccgaaggagaaaaag-3′; | (SEQ ID NO. 103) and |
| 5′-gcctcagtcctgttctcttcc-3′. | (SEQ ID NO. 104) |

Primer extension was then performed with Superscript II reverse transcriptase. In this reaction 4 pmol of labelled primer was combined with 300 nmol of RNA and denatured at 90° C. for 2 min. The primer was then allowed to anneal slowly between 65° C.-45° C. before adding the first strand buffer; dithiothreitol, deoxynucleotides and enzyme. This mix (20 μl) was incubated at 45° C. for 1 hr, before being stopped by placing the reaction on ice. Samples were placed in an equal volume of stop buffer and then run on a 6% polyacrylamide gel. Sequencing was performed by primer extension on the double stranded cDNA template in the presence of chain terminating dideoxynucleotides (ddNTP)(Sambrook et al., 1989). The sequence was used as a guide to attribute cleavage bands to specific DNAzymes. The relative cleavage strength of each DNAzyme was determined by intensity of the cleavage products. DNAzymes were ranked according to their cleavage ability at lowest concentration (5 nM). In vitro selection of bcl-2 DNAzymes was achieved by incubating Bcl-2 DNAzymes with its RNA substrate for 60 minutes in the presence of 10 mM Mg²⁺ at 37° C. Primer extension was performed using the sequence-specific primers along the bcl-2 mRNA. The reactions were analysed alongside with DNA sequencing on a polyacrylamide gel. In vitro selection of bcl-xL DNAzymes was achieved by incubating Bcl-xl DNAzymes with its RNA substrate for 60 minutes in the presence of 10 mM Mg²⁺ at 37° C. Primer extension was performed using the sequence-specific primers along the bcl-xl mRNA. The reactions were analysed alongside with DNA sequencing on a polyacrylamide gel.

### Example 3

Porphyrin-Mediated DNAzyme Uptake in Cancer Cells

To test the selected DNAzymes in cell culture systems, a prostate cancer cell line PC3 was initially used to examine their efficacy in down-re gulation of bcl-2 and bcl-xL gene expression and impact on cellular functions. To facilitate delivery of DNAzyme oligonucleotides into cells, a cationic porphyrin, tetra meso-(4-methylpyridyl) porphyrine (TMP), was used as a transfection reagent for intracellular delivery (Benimetskaya et al., 1998).

Chemical Modification of DNAzymes:

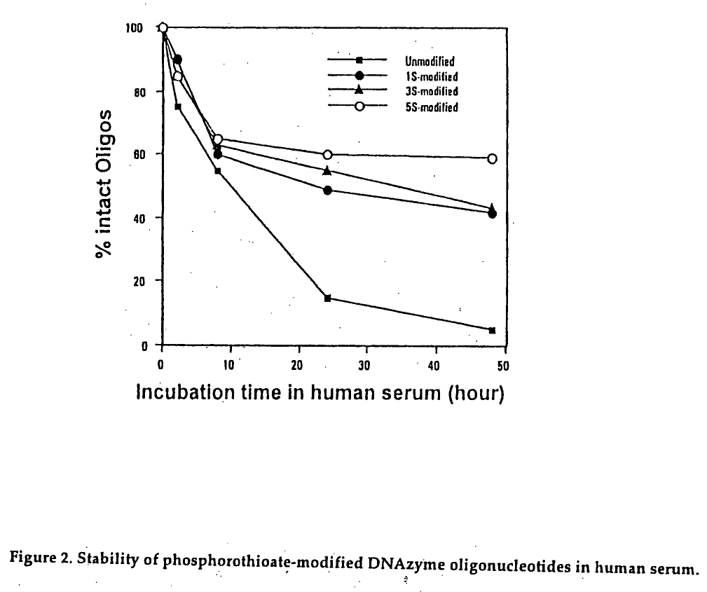

To increase DNAzyme stability in cells, two phosphorothioate linkages were incorporated into each of the arms in DNAzymes (PS-Dz)(Wagner et al. 1995). This has been shown to increase the DNAzyme stability significantly in human serum, while there was no marked effect on the DNAzyme cleavage activity (FIG. 2).

DNAzyme Transfection Efficiency:

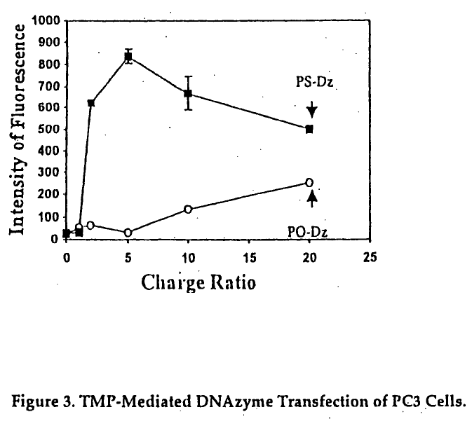

1.2×10⁶ cells were seeded in a 100-mm culture dish and incubated at 37° C., 5% CO₂ overnight. The cells were transfected with an FITC-labelled DNAzyme that was complexed with TMP at a charge ratio of 3 (+/−). The transfected cells were analysed using FACS and fluorescent microscopy. As shown in FIG. 3, a more efficient delivery was observed when POS-Dz was complexed with TMP, compared with normal phosphodiester DNAzyme (PO-Dz). In addition, nuclear delivery of the DNAzymes (FITC-labelled) was evident.

### Example 4

Suppression of bcl-2 and bcl-xL Expression in Cancer Cells.

From the in vitro multiplex selection, 26 DNAzymes against bcl-2 (26/55) and 16 DNAzymes against bcl-xL (16/26) were shown to be efficient cleavers of their corresponding substrates. The modified version of these molecules were then tested for their ability to down regulate the bcl-2 and bcl-xL expression in cells. The assays were performed in PC3 cells (a prostate cancer cell line). The cells were transfected with 2 μM DNAzyme complexed with TMP at a charge ratio of 3. After overnight incubation, cells were subject to either protein (Western blot) or RNA (Ribonuclease protection assay) analyses (Sambrook et al 1989).

Effect of Bcl-2 DNAzymes on bcl-2 Expression in PC3 Cells:

All 26 DNAzymes were tested in transfection assay for their activity by Western blots. Five out of 26 DNAzymes showed a consistent inhibitory effect on the bcl-2 protein level (Table 3). The effect of bcl-2 DNAzymes on expression of the bcl-2 gene family was determined by transfecting five active DNAzymes into PC3 cells (2 μM). DT907 was used as an inactive DNAzyme control. Antibodies to Bcl-2, Bcl-xL, Bax and β-actin were used respectively to detect the corresponding proteins. While TMP alone and inactive DNAzyme control did not show any effect, all the five DNAzymes suppressed Bcl-2 level significantly. These DNAzymes had no effect on either other members of the bcl-2 gene family such as Bcl-xL and Bax, or house keeping gene β-actin.

TABLE 3

Active bcl-2 DNAzymes identified in
Western analyses.

Target

| | | |
|---|---|---|
| DNAzyme | DNAzyme sequence | sites* |

| | | |
|---|---|---|
| DT895 | Cccagttcaggctagctacaacgacccgtccct | 455 |
| DT902 | Agataggcaggctagctacaacgaccagggtga | 729 |
| DT908 | Tttttcccaggctagctacaacgacgctgtcct | 1432 |
| DT910 | Ccctgttgaggctagctacaacgacatccctgg | 1806 |
| DT912 | Cacagccaaggctagctacaacgagtgccatgt | 2093 |

*indicates the cleavage site on human bcl-2 mRNA sequence.

Effect of bcl-xL DNAzymes on the bcl-xL Expression:

After screening all 16 DNAzymes, three DNAzymes, DT882, DT883 and DT884, exhibited a very strong inhibitory effect on bcl-xL protein expression (Table 4). Suppression of bcl-xL protein level by bcl-xL DNAzymes was determined by transfecting three active DNAzymes into PC3 cells (2 μM). DT867 and 880 were used as inactive DNAzyme controls; and DT888 as an antisense control. Antibodies to Bcl-2 and β-actin were used respectively to detect the corresponding proteins. DT 880 and DT867 were inactive DNAzymes in this screening. The effect in PC3 cells was further confirmed using an RNase protection assay (RPA) of bcl-xL DNAzyme. In the RNase protection assay, DNAzymes were complexed with TMP at a charge ratio of 3 and transfected into PC3 cells. Cellular RNA was extracted from the transfected cells and used for RPA analysis. Apoptosis related riboprobe set was generated from a Pharmingen kit.

TABLE 4

Active bcl-xL DNAzymes identified in
Western analyses.

Target

| | | |
|---|---|---|
| DNAzyme | DNAzyme sequence | sites* |

| | | |
|---|---|---|
| DT882 | Tttttataaggctagctacaacgaagggatggg | 126 |
| DT883 | Acatttttaggctagctacaacgaaatagggat | 129 |
| DT884 | Tctgagacaggctagctacaacgattttataat | 135 |

*indicates the cleavage site on human bcl-xL mRNA sequence.

### Example 5

Bcl-2 and bcl-xL Specific DNAzyme-Mediated Effect on Cell Cycle

Following the test of the DNAzymes in Western and RPA assays, some of the active molecules were further examined for their effect on cell cycle as an indication of apoptotic response. Two most active DNAzymes were chosen in FACS assay. These were DT 895 (a bcl-2 DNAzyme) and DT882 (a bcl-xL DNAzyme). In the assay, same transfection procedure as in Western assay was used, except those cells were subject to PI staining after the overnight incubation with the DNAzymes. Table 5 clearly showed that there was a substantial increase in sub G1 population in the DNAzyme treated cells (DT895 12.82% and DT882 23.17% respectively), indicating that the cells treated with anti-bcl-2 and bcl-xL DNAzymes were provoked to undergo apoptosis.

TABLE 5
Cell cycle analysis of DNAzyme-transfected PC3 cells.

| Treatment | % Sub-G1 population |
|---|---|
| PC3 | 0.63 |
| TMP | 2.2 |
| Bcl-2 DNAzyme 895 | 12.82 |
| Bcl-xL DNAzyme 882 | 23.17 |
| Inactive control | 1.62 |

### Example 6

Effect of the Bcl-xL DNAzyme on Cytochrome C Release

Cytochrome c is a well-characterised mobile electron transport protein essential to energy conversion in all-aerobic organisms. In mammalian cells, this highly conserved protein is normally localised to the mitochondrial intermembrane space. More recent studies have identified cytosolic cytochrome c as a factor necessary for activation of apoptosis. During apoptosis, cytochrome c is translocated from the mitochondrial membrane to the cytosol, where it is required for activation of caspase-3 (CPP32). It has been reported that the translocation of cytochrome c can be blocked by overexpression of Bcl-2 or Bcl-xL. Based on this, the measurement of CytoC release from cells would be an ideal assay to determine the bcl-xL DNAzyme effect on the early events of apoptosis caused by down-regulation of bcl-xL. After transfection of PC3 cells with bcl-xL DNAzymes, the proteins from the cytoplasmic fraction were extracted and subjected to Western analysis. Studies by the applicants determined that Bcl-xL DNAzyme-mediated down-regulation of bcl-xL and increased release of Cytochrome. C. In these studies PC3 cells were transfected with 2 μM DNAzyme complexed with TMP. Western analyses were performed using the antibodies to Bcl-xL and Cytochrome C. DNAzyme-mediated reduction of bcl-xL in PC3 cells led to an increased release of CytoC. This result not only confirmed previous data from cell cycle analysis, but also validated the specificity of the DNAzyme against apoptotic pathway in PC3 cells.

### Example 7

Chemosensitization of PC3 Cells with Anti-bcl-xL DNAzymes

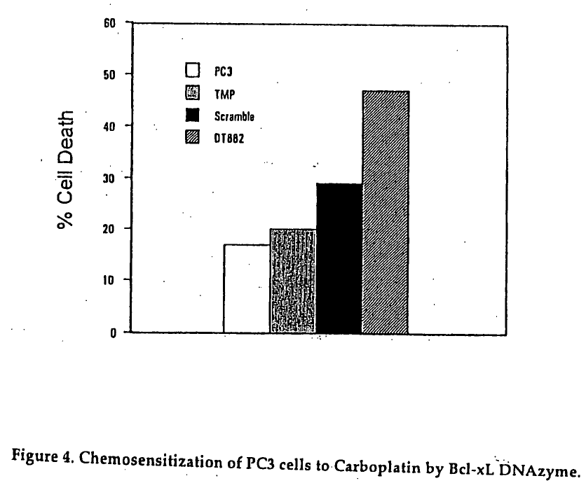

The Bcl-xL protein has been shown in a number of cell lines to be a potent protector of cellular apoptosis induced by anti-neoplastic agents. Thus an efficient DNAzyme that decreased Bcl-xL expression in PC3 cells would sensitise them to the effect of cytotoxic therapy. To test this, cell survival was measured using MTS assays in PC3 cells treated with either DNAzyme alone or DNAzyme plus anti-cancer agents such as Carboplatin. The result in FIG. 4 demonstrated that the anti-bcl-xL DNAzyme DT882 sensitised PC3 cells to Carboplatin treatment at 5 μM. This sensitization led to an increase of cell death from 17% when only Carboplatin was used, to about 50% cell death when the DNAzyme and Carboplatin were combined.

### Example 8

Use of Anti-bcl-2 and bcl-xL DNAzymes in Other Tumour Cell Lines

High level expression of Bcl-2 and Bcl-xL has been found in various types of cancers. In addition to the efficacy of the DNAzymes shown in Prostate cancer cell lines (PC3 and DU145), further cell-based assays were performed to explore the therapeutic potential of the anti-bcl-2 and bcl-xL DNAzymes in vivo. Several cell lines of various cancer types have been used to validate the DNAzyme efficacy in the different settings. These are T24, bladder cancer; HCT116, colon cancer; and A549, lung carcinoma.

To analyse inhibition of Bcl-2 expression in different tumour cells by bcl-2 DNAzymes, T24 (bladder), A549 (lung) and HCT116 (colon) cells were treated with 2 μM DNAzyme complexed with TMP at a charge ratio of 3. After 24 hours post transfection, the cellular protein was extracted and immunoblotted with bcl-2 antibody or β-actin antibody. Inhibition of Bcl-xL expression in different tumour cells by bcl-xL DNAzyme was also investigated using T24 (bladder), A549 (lung) and HCT116 (colon) cells treated with 2 μM DNAzyme complexed with TMP as described and immunoblotted with bcl-xL antibody or β-actin antibody. These studies show that both anti-bcl-2 and anti-bcl-xL DNAzymes reduced the level of their respective gene expression in all the cell lines tested.

### Example 9

Chemosensitization in Human Tumour Xenograph Models by Anti-Bcl-xL DNAzyme DT882.

In order to demonstrate that down-regulation of the bcl-2 gene family results in Chemosensitization of tumour cells to anticancer drugs, murine models with human PC3 prostate cancer and MDA-MB-231 breast cancer xenograph were used to determine if the sensitivity to the chemotherapeutic is enhanced.

In the experiments, four groups of mice (8 mice per group) (Saline, DNAzyme, Taxol, Taxol+DNAzyme) were employed At day 1: acclimatised nude male Balb/C athymic mice were injected with 1×10⁶ tumor cells suspended in 0.1 ml Matrigel in the right hind leg under methoxyfluorane anesthesia. Tumour growth is measured twice weekly using digital callipers and tumour volume is calculated using the (l×w×h×π/6) formula. When tumours reach an average volume of 100-200 mm³, an Alzet osmotic pump, which were used as a delivery vehicle for DNAzyme oligonucleotides in tumour bearing mice, was surgically implanted in the peritoneum of the mouse via the abdominal route. The Alzet model1002 pump is a capsule shaped pump (1.5×0.6 cm) and delivers a total volume of 0.5 nm at a rate of 0.25 μl/hr over a period of 14 days. The pump was filled with a saline solution containing DNAzyme oligonucleotide, which resulted in a dose rate of 12.5 mg/kg/d. Some mice will receive 25 mg/kg Taxol by intraperitoneal route in a 200 μl injection once weekly post-surgery for the duration of the study.

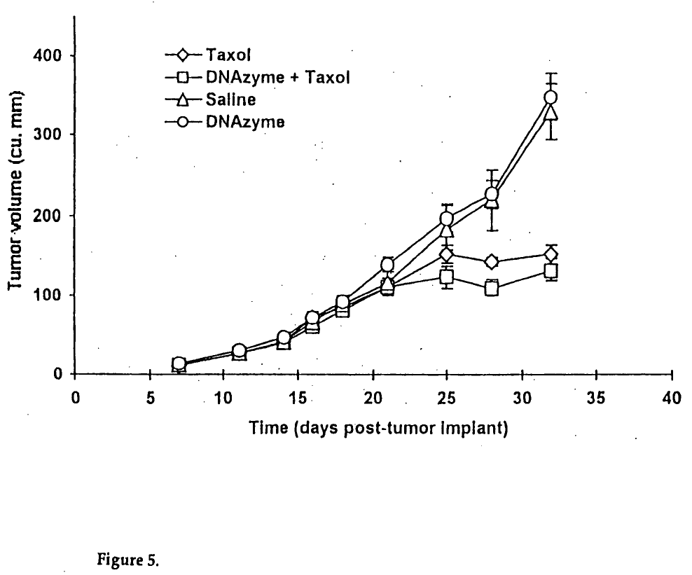

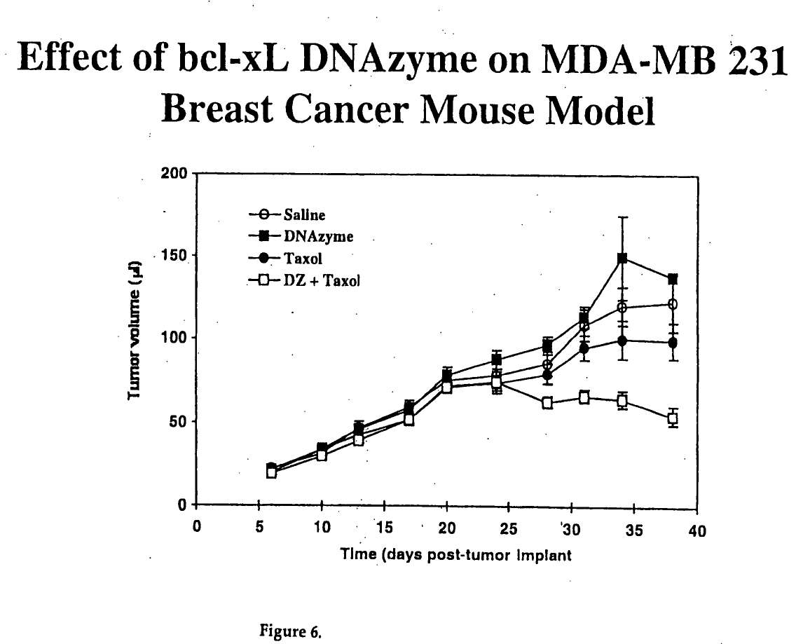

As shown in FIGS. 5 and 6, combination of DNAzyme and Taxol treatment markedly inhibited both PC3 and MDA-MB-231 tumour growth compared with the groups of DNAzyme alone or Taxol alone.

### Example 10

Chemosensitization in Human Tumour Xenograph Models by Anti-Bcl-2 DNAzyme. DT912.

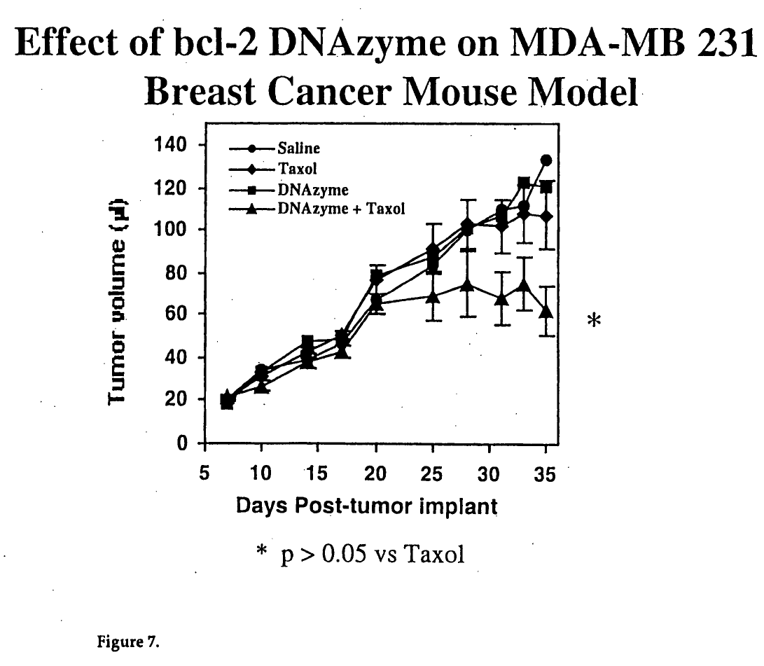

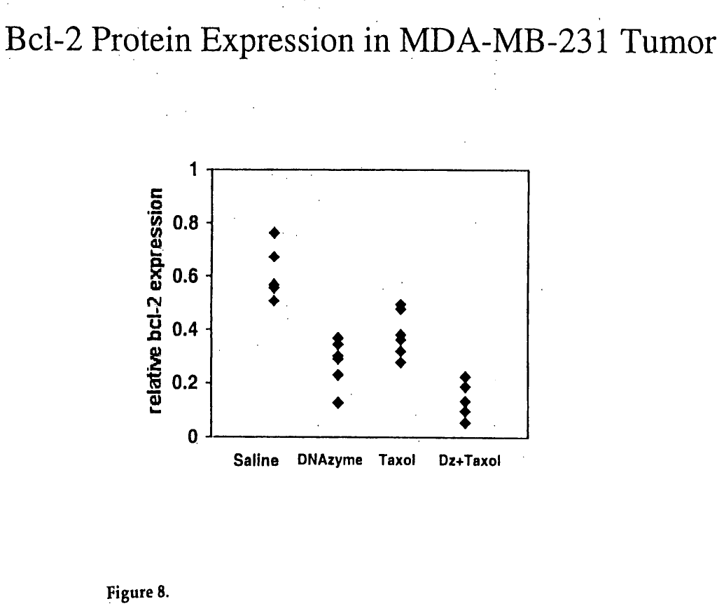

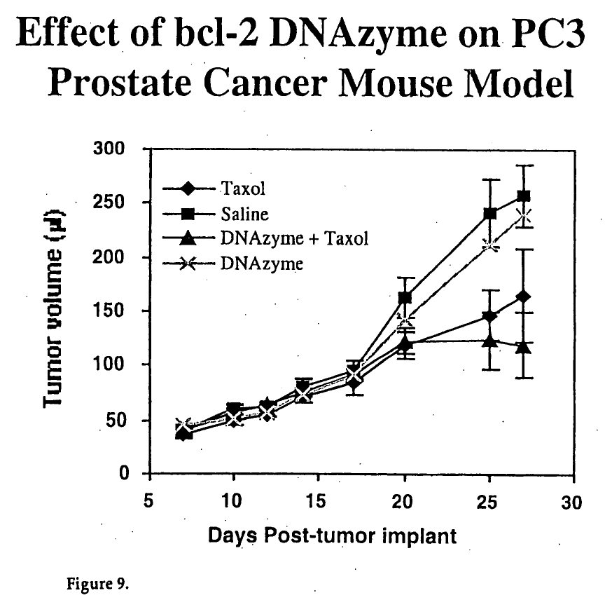

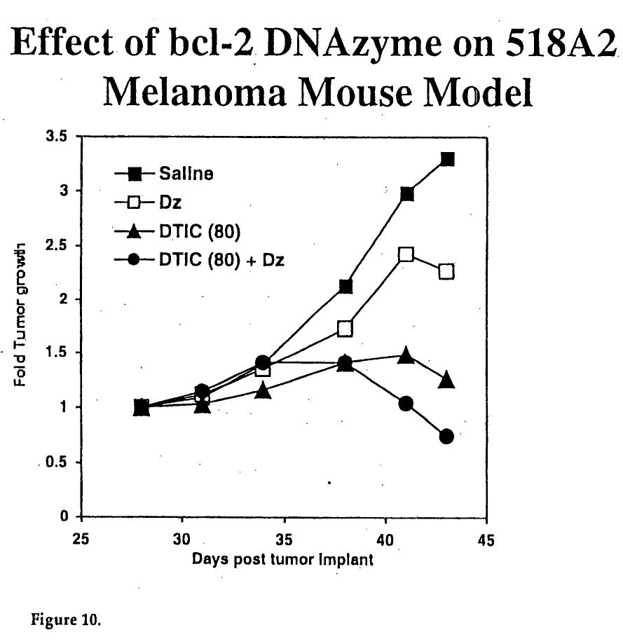

As described in Example 9, both prostate and breast cancer models were also used in testing the bcl-2 DNAzyme efficacy. In addition, a human melanoma model (518A2) was further used to determine the effect of the treatment of bcl-2, combined with Dacarbazine (DTIC), on the tumor growth. As shown in FIGS. 7, 9 and 10, the anti-bcl-2 DNAzyme DT912 could sensitise all three tumours to chemotherapeutic treatment and this effect was closely related to the down-regulation of the bcl-2 protein level (FIG. 8).

### Example 11

Accessibility and Efficacy of Antisense Oligonucleotides Cannot be Correlated to DNAzyme Targeting.

The protooncogene c-myb plays an important role in proliferation and differentiation of haematopoietic cells. C-myb protein levels vary according to the level of differentiation of normal haematopoietic cells with low protein expression detected in terminally differentiated cells. In leukemia cells where there is rapid proliferation of myeloid precursors, c-myb has often been found to be overexpressed. In the literatures, it has been shown that use of antisense oligonucleotides could inhibit the c-myb expression in vitro and led to suppress leukemia development. Against same regions targeted by antisense oligonucleotides, DNAzymes were designed and tested in leukemia cell cultures.

In the experiments, K562 cells were transfected with 2 μM oligo complexed with TMP at a charge ratio (+/−) of 5 on Days 0 and 1. Cellular proteins were extracted on Day 2 and analysed by Western using a monoclonal antibody to c-Myb. Inhibition of c-Myb protein expression by antisense and DNAzymes was determined by western blot analysis. Two antisense oligonucleotides DT860 (gtgccggggtcttcgggc,) (SEQ ID NO. 105) and DT1019 (gctttgcgatttctg;)(SEQ ID NO.106), consistently showed efficacy in inhibiting c-Myb protein expression, while none of the DNAzymes corresponding to these sites could effectively reduce c-Myb protein levels

This example clearly demonstrates that the different structures and conformations of oligonucleotides and DNAzymes results in these two molecules having different accessibility to their target RNA. Thus, the effect of the one type of agent is not a predictor of the activity of another type.

TABLE 6
Sequence ID Nos and description.

| SEQUENCE | | Database |
|---|---|---|
| ID | | Accession |
| NO. | Description | Number |
| 1 | DNAzyme catalytic domain | |
| 2 | Bcl-2 CDS | M14745 |
| 3 | Bcl-xL CDS | Z23115 |
| 4 | Bcl-w gene | NM_004050 |
| 5 | Bfl-1 gene | U27467 |
| 6 | Mcl-1 gene | AF147742 |
| 7-61 | Bcl-2 DNAzymes | |
| 62-87 | Bcl-xL DNAzymes | |
| 88 | Bcl-2 A1 gene | NM_004049 |
| 89 | BRAG-1 gene | S82185 |
| 90-100 | bcl-2 cleavage detection primers | |
| 101-104 | bcl-xL cleavage detection primers | |
| 105 | Antisense oligonucleotide | |
| 106 | Antisense oligonucleotide | |

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### REFERENCES

Adams, J. M., and Cory, S. (1998). The Bcl-2 protein family: arbiters of cell survival. Science 281, 1322-6.
Ashkenazi, A., and Dixit, V. M. (1998). Death receptors: signaling and modulation. Science 281, 1305-8.
Benimetskaya, L., Takle, G. B., Vilenchik, M., Lebedeva, I., Miller, P., and Stein, C. A. (1998). Cationic porphyrins: novel delivery vehicles for antisense oligodeoxynucleotides. Nucleic Acids Res 26, 5310-7.
Breaker R R and Joyce G F (1994) A DNA enzyme that cleaves RNA. Chem Biol 1:223-229.
Breaker R R and Joyce G F (1995) A DNA enzyme with Mg(2⁺)-dependent RNA phosphoesterase activity. Chem Biol 2:655-660.
Cairns, M. J., Hopkins, T. M., Witherington, C., Wang, L., and Sun, L. Q. (1999). Target site selection for an RNA-cleaving catalytic DNA [see comments]. Nat Biotechnol 17, 480-6.
Carni N, Balkhi S R and Breaker R R. (1998) Cleaving DNA with DNA. Proc Natl Acad Sci USA. 95:223S2237.
Ellis, R. E., Yuan, J. Y., and Horvitz, H. R. (1991). Mechanisms and functions of cell death. Annu Rev Cell Biol 7, 663-98.
Gewirtz, A. M., Sokol, D. L., and Ratajczak, M. Z. (1998). Nucleic acid therapeutics: state of the art and future prospects. Blood 92, 712-36.
Haseloff J and Gerlach W L (1988) Simple RNA enzymes with new and highly specific endoribonuclease activities Nature 334:585-591.
Jansen; B., Schlagbauer-Wadl, H., Brown, B. D., Bryan, R. N., van Elsas, A., Muller, M., Wolff, K., Eichler, H. G., and Pehamberger, H. (1998); Bcl-2 antisense therapy chemosensitizes human melanoma in SCID mice. Nat Med 4, 232-4.
Kashani-Sabet M, Funato T et al (1992) Reversal of malignant phenotype by an anti-ras ribozyme. Antisense Res Dev 2:3-15.
Koizurni M, Kamiya H and Ohtsuka E (1993) Inhibition of c-Ha-ras gene expression by hammerhead ribozymes containing a stable C(UUCG)G hairpin loop. Biol Pharm Bull 16:879-883.
Pan T (1997) Novel and variant ribozymes obtained through in vitro selection. Curr Opin Chem Biol. 1:17-25.
Raillard S A and Joyce G F (1996) Targeting sites within HIV-1 cDNA with a DNA-cleaving ribozyme. Biochemistry. 35(36):11693-701
Reed, J. C. (1998). Bcl-2 family proteins. Oncogene 17, 3225-36.
Reed, J. C. (2000). Mechanisms of Apoptosis. Am J Pathol 157, 1415-1430.
Sambrook J., Fritsch E. F. and Maniatis T., 1989 Molecular Cloning, a laboratory manual 2^{nd} ed. Cold Spring Harbour Laboratory Press.
Santiago, F. S., Lowe, H. C., Kavurma, M. M., Chesterman, C. N., Baker, A., Atkins, D. G., and Khachigian, L. M. (1999). New DNA enzyme targeting Egr-1 mRNA inhibits vascular smooth muscle proliferation and regrowth after injury [published erratum appears in Nat Med 5(12): 1438. Nat Med 5, 1264-9.
Santoro, S. W., and Joyce, G. F. (1997). A general purpose RNA-cleaving DNA enzyme. Proc Natl Acad Sci USA 94, 4262-6.
Santoro S W and Joyce G F (1998) Mechanism and utility of an RNA-cleaving DNA enzyme. Biochemistry37:13330-13342.
Sugimoto, N., Nakano, S., Katoh, M., Matsumura, A., Nakamuta, H., Ohmichi, T., Yoneyama, M., and Sasaki, M. (1995). Thermodynamic parameters to predict stability of RNA/DNA hybrid duplexes. Biochemistry 34, 11211-6.
Sun, L. Q., Cairns, M. J., Gerlach, W. L., Witherington, C., Wang, L., and King, A. (1999). Suppression of smooth muscle cell proliferation by a c-myc RNA-cleaving deoxyribozyme. J Biol Chem 274, 17236-41.
Tsang J and Joyce G F (1996) Specialization of the DNA-cleaving activity of a group I ribozyme through in vitro evolution.
J. Mol. Biol. 262:31-42.
Vocero-Akbani, A. M., Heyden, N. V., Lissy, N. A., Ratner, L., and Dowdy, S. F. (1999). Killing HIV-infected cells by transduction with an HIV protease-activated caspase-3 protein [see comments]. Nat Med 5, 29-33.
Warashina, M., Kuwabara, T., Nakamatsu, Y., and Taira, K. (1999). Extremely high and specific activity of DNA enzymes in cells with a Philadelphia chromosome. Chem Biol 6, 237-50.
Wagner, R. W. (1995) Nature Med 1:1116-1118.
Waters, J. S., Webb, A., Cunningham, D., Clarke, P. A., Raynaud, F., di Stefano, F., and Cotter, F. E. (2000). Phase I clinical and pharmacokinetic study of bcl-2 antisense oligonucleotide therapy in patients with non-Hodgkin's lymphoma [see comments]. J Clin Oncol 18, 1812-23.
Wu, Y., Yu, L., McMahon, R., Rossi, J. J., Forman, S. J., and Snyder, D. S. (1999). Inhibition of bcr-abl oncogene expression by novel deoxyribozymes (DNAzymes). Hum Gene Ther 10, 2847-57.
Yakovlev, A. G., Knoblach, S. M., Fan, L., Fox, G. B., Goodnight, R., and Faden, A. I. (1997); Activation of CPP32-like caspases contributes to neuronal apoptosis and neurological dysfunction after traumatic brain injury. J Neurosci 17, 7415-24.
Zangemeister-Wittke, U., Leech, S. H., Olie, R. A., Simoes-Wust, A. P., Gautschi, O., Luedke, G. H., Natt, F., Haner, R., Martin, P., Hall, J., Nalin, C. M., and Stahel, R. A. (2000). A novel bispecific antisense oligonucleotide inhibiting both bcl-2 and bcl-xL expression efficiently induces apoptosis in tumour cells [In Process Citation]. Clin Cancer Res 6, 2547-55.

1. A DNAzyme which specifically cleaves mRNA transcribed from a member of the bcl-2 gene family selected from the group consisting of bcl-2, bcl-xl, bcl-w, bfl-1, brag-1, Mcl-1 and A1, the DNAzyme comprising (a) a catalytic domain that has the nucleotide sequence GGCTAGCTACAACGA and cleaves mRNA at any purine:pyrimidine cleavage site at which it is directed, (b) a binding domain contiguous with the 5′ end of the catalytic domain, and (c) another binding domain contiguous with the 3′ end of the catalytic domain, wherein the binding domains are complementary to, and therefore hybridise with, the two regions immediately flanking the purine residue of the cleavage site within the bcl-2 gene family mRNA, at which DNAzyme-catalysed cleavage is desired, and wherein each binding domain is at least six nucleotides in length, and both binding domains have a combined total length of at least 14 nucleotides.

2. A DNAzyme according to claim 1 wherein the DNAzyme is 29 to 39 nucleotides in length.

3. A DNAzyme according to claim 1 wherein the bcl-2 gene family member is bcl-2 or bcl-xl.

4. A DNAzyme according to claim 1 selected from the group consisting of those listed in SEQ ID NOS. 7 to 61.

5. A DNAzyme according to claim 1 wherein the DNAzyme cleaves bcl-2 mRNA at position 455, 729, 1432, 1806 or 2093.

6. A DNAzyme according to claim 1 wherein the sequence of the DNAzyme is set out in SEQ ID NOS 24, 45, 53, 55 or 57.

7. A DNAzyme according to claim 1 selected from the group consisting of those listed in SEQ ID NOS. 62 to 87.

8. A DNAzyme according to claim 1 wherein the DNAzyme cleaves bcl-xl mRNA at position 126, 129 or 135.

9. A DNAzyme according to claim 1 wherein the sequence of the DNAzyme sequence is set out in SEQ ID NOS 82, 83 or 84.

10. A DNAzyme according to claim 1 wherein 1 to 6 phosphorothioate linkages are introduced into each of the 5′ and 3′ ends of the DNAzymes.

11. A DNAzyme according to claim 1 wherein the DNAzyme comprises at least one modification selected from the group consisting of 3′-3′ inversion, N3′-P5′ phosphoramidate linkages, peptide-nucleic acid linkages, and 2′-O-methyl.

12. A pharmaceutical composition comprising a pharmaceutically acceptable carrier and at least one DNAzyme according to claim 1.

13. A pharmaceutical composition according to claim 13 wherein the composition further comprises at least one chemotherapeutic agent selected from the group consisting of taxol, daunorubicin, dacitinomycin, doxorubicin, bleomycin, mitomycin, nitrogen mustard, chlorambucil, melphalan, cyclophosphamide, 6-mercaptopurine, 6-thioguanine, cytarabine, 5-flurouracil, floxuridine, methotrexate, colchicine, vincristine, vinlastin, etoposide and cisplatin.

14. A method of treating tumours in a subject, the method comprising administering to the subject a composition according to claim 13.

15. A method of enhancing the sensitivity of malignant or virus infected cells to therapy, the method comprising modulating expression level of a member of the bcl-2 gene family selected from the group consisting of bcl-2, bcl-xl, bcl-w, bfl-1, brag-1, Mcl-1 and A1 using a DNAzyme according to claim 1.

16. A method of treating tumours in a subject, the method comprising administering to the subject a first composition comprising at least one DNAzyme according to claim 1 and a second composition comprising an anticancer agent.

17. A method as claimed in claim 16 in which the anticancer agent is selected from the group consisting of tazol, daunorubicin, dacitinomycin, doxorubicin, bleomycin, mitomycin, nitrogen mustard, chlorambucil, melphalan, cyclophosphamide, 6-mercaptopurine, 6-thioguanine, cytarabine, 5-flurouracil, floxuridine, methotrexate, colchicine, vincristine, vinlastin, etoposide and cisplatin.
